SEQUENCE LISTING (11 sequences)

SEQ ID NO: 1 - DNA, 29 bp, Escherichia coli
gacagaaaaagtaaaaaacgcggagcaat

SEQ ID NO: 2 - DNA, 31 bp, Escherichia coli
aacgcgaagtagtaataaataaatgggagta

SEQ ID NO: 3 - protein, 116 aa, Bacillus subtilis
MIVKRGDVYFADLSPVVGSEQGGVRPVLVIQNDIGNRFSPTAIVAAITAQIQKAKLPTHVEIDAKRYGFERDSVILLEQIRTIDKQRLTDKITHLDDEMMDKVDEALQISLALIDF

SEQ ID NO: 4 - protein, 116 aa, Bacillus anthracis
MIVKRGDVYFADLSPVVGSEQCGVRPVLVIQNDIGNRFSPTVIVAAITAQIQKAKLPTHVEIDAKKYGFERDSVILLEQIRTIDKQRLTDKITHLDEVMMIRVDEALQISLGLIDF

SEQ ID NO: 5 - protein, 136 aa, Staphylococcus aureus
MIRRGDVYLADLSPVQGSEQGGVRPVVIIQNDTGNKYSPTVIVAAITGRINKAKIPTHVEIEKKKYKLDKDSVILLEQIRTLDKKRLKEKLTYLSDDKMKEVDNALMISLGLNAVAQPEKLGVYYMYFSEINKILI

SEQ ID NO: 6 - protein, 111 aa, Escherichia coli
MVSRYVPDMGDLIWVDFDPTKGSEQAGHRPAVVLSPFMYNNKTGMCLCVPCTTQSKGYPFEVVLSGQERDGVALADQVKSIAWRARGATKKGTVAPEELQLIKAKINVLIG

SEQ ID NO: 7 - DNA, 79 bp, Artificial
  GFP fragments in a plasmid
ctggctagacaccatggccagcaaaggagaagaacttttcactggcatggatgagctctacaaaagcggttccggaccg

SEQ ID NO: 8 - DNA, 18 bp, Artificial
  His tag for Figure 20
caccaccaccaccaccac

SEQ ID NO: 9 - DNA, 12 bp, Artificial
  chimeric cleavage substrate
gatauacatatg

SEQ ID NO: 10 - DNA, 12 bp, Artificial
  chimeric cleavage substrate
gatauacgtatg

SEQ ID NO: 11 - DNA, 12 bp, Artificial
  chimeric cleavage substrate
gatauacctatg

(19) United States
(12) Patent Application Publication
(10) Pub. No.: US 2010/0234287 A1
(43) Pub. Date: Sep. 16, 2010

(54) Targeting Bacterial Suicide Pathways for the Development of Novel Antibiotics

(75) Inventors: Masayori Inouye, New Brunswick, NJ (US); Nancy Woychik, Morristown, NJ (US); Koichi Inoue, East Brunswick, NJ (US)

Correspondence Address: PATENT DOCKET ADMINISTRATOR;LOWENSTEIN SANDLER PC, 65 LIVINGSTON AVENUE, ROSELAND, NJ, 07068, US

(73) Assignee: UNIVERSITY OF MEDICINE AND DENTISTRY OF NEW JERSEY, Somerset, NJ (US)

(21) Appl. No.: 12/293,838

(22) PCT Filed: Mar. 22, 2007
(86) PCT No.: PCT/US2007/064739

§ 371 Date: Dec. 16, 2008

Related U.S. Application Data
(60) Provisional application No. 60/784,776, filed on Mar. 22, 2006.

(51) Int. Cl. A61K 38/00; C12Q 1/68; C12N 1/20; A61K 38/16; C07K 2/00; C07K 14/00

(52) U.S. Cl. 514/12; 435/6; 435/252.1; 514/2; 530/300; 530/350

(57) ABSTRACT

The invention provides methods for identifying an agent which prevents or partially prevents an antitoxin from forming a complex with its cognate toxin, comprising contacting a potential agent with a labeled substrate in solution, whereby detection of the label indicates presence of an agent that prevents an antitoxin from forming complex with a toxin. The invention also provides agents capable of interfering with formation of a toxin-antitoxin complex. Such agents act as novel, non-conventional antibiotics against human pathogenic bacteria.

### CROSS-REFERENCE TO RELATED APPLICATION

This application claims priority to U.S. Provisional Application No. 60/784,776 entitled “Targeting Bacterial Suicide Pathways for the Development of Novel Antibiotics” by Inouye et al., filed on Mar. 22, 2006. The entire disclosure of this application is incorporated herein by reference.

### FIELD OF THE INVENTION

The present invention relates to systems for enhancing the production and solubility of proteins.

### BACKGROUND OF THE INVENTION

This invention relates to a novel approach to search for new antibiotics, which is not based on the conventional target screening methods. This approach takes advantage of the bacterial suicide systems, which prevail in all bacterial species except for symbiotic bacteria.

Antibiotics in general target the biosynthetic pathways in bacteria such as cell wall synthesis, DNA replication, RNA synthesis, protein synthesis and synthesis of essential small molecules such as amino acids, nucleotides and co-factors. As a result of inhibition of a target pathway by an antibiotic, bacterial cell growth is inhibited, which in many cases leads to cell death.

Bacteria are generally equipped with the so-called toxin-antitoxin (TA) or “suicide” gene systems, which are considered to play important roles in growth regulation, cell death and dormancy under stress conditions. Under normal growth conditions, a toxin forms a stable complex with its cognate antitoxin encoded from the same operon (TA operon), thus the toxin is incapacitated for acting on its cellular target. However, under stress conditions, labile antitoxins are rapidly degraded with concomitant release of free toxins in the cytoplasm, which then exert their toxic effect on specific cellular targets.

The number of toxin or suicide genes present on the bacterial genomes widely varies; Escherichia coli typically contains six independent TA operons, each encoding a pair of an antitoxin and its cognate toxin, while Mycobacterium tuberculosis contains approximately forty such operons. All the pathogenic bacterial genomes sequenced to date indeed contain one or more TA operons except for bacteria that live obligatorily with host cells such as Chlamydia and Mycoplasm. Out of six TA operons in E. coli, three have been well characterized; RelE is a ribosome-associating factor that stimulates ribosomal endo-ribonuclease activity, and MazF and ChpBK act as sequence-specific endo-ribonucleases, termed mRNA interferases (MIase). It has been demonstrated that MazF, when induced, cleaves cellular mRNAs at ACA sequences thereby effectively inhibiting cellular protein synthesis and thus cell growth. MazF forms a stable complex with its antitoxin, MazE, and the X-ray structure of the MazF-MazE complex has been determined. Since the TA complexes are not toxic to the cells, they are well expressed in E. coli and are readily purified with a very high yield. Recently, the X-ray structures of the RelE-RelB and the YoeB-YefM complexes have also been determined, revealing how toxins and antitoxins interact in the TA complexes.

Most bacteria contain a number of toxin or “suicide” genes in their genomes. Importantly, the toxins produced from these genes are neither intended to kill other bacteria in their habitats nor to kill animal cells in the process of infection. Instead, they are produced intracellularly and are toxic to themselves. Recent developments in this new field have provided many intriguing insights into the role of these toxins in bacterial physiology, persistence in multi-drug resistance, pathogenicity, biofilm formation and evolution. It is now evident that the study of these toxins has very important implications in infectious diseases and medical sciences. Since most of these toxins are co-transcribed with their cognate antitoxins in an operon (thus termed as toxin-antitoxin or TA operons), and they form a stable complex in the cell under normal growth conditions, the toxic effect of these toxins is not typically exerted (Bayles, 2003; Engelberg-Kulka et al., 2004; Hayes, 2003; Rice and Bayles, 2003). However, since the stability of antitoxins is much less than that of their cognate toxins, any stress causing cellular damage or growth inhibition affects the balance between toxin and antitoxin in the cell, leading to release of toxins in the cell. Although much debated, it is most reasonable to consider that these toxins encoded from the TA operons function in two different ways depending upon the nature of the stress. One is to regulate the growth rate by inhibiting a particular cellular function such as DNA replication and protein synthesis. Under extensive stress, at which the amount of toxins exceeds the antitoxins, cell growth may be completely arrested. This role of TA toxins in growth regulation is likely to be their primary function. However, their second role is suicidal, that is to kill their own host cells. Under certain conditions, TA toxins may function to eliminate cells that are highly damaged (for example, DNA damage or phage infection) to maintain a healthy population. The TA operons are also often found in plasmids, which play a role in killing the cells that have lost plasmids after cell division; a phenomenon known as post-segregational killing. Therefore, TA toxins are primarily bacteriostatic, but not bactericidal (Gerdes et al., 2005) but under certain conditions, cells may reach a point of no return resulting in cell death (Amitai et al, 2004). Recently, Engelberg-Kulka proposed that MazF, an E. coli toxin, is not an executioner of cell death but is rather a mediator that activates downstream systems (Engelberg-Kulka et al., 2005).

To date, a number of TA modules have been studied in some detail—the bacteriophage encoded phd-doe module (Gazit and Sauer, 1999), plasmid encoded kis-kid (Hargreaves et al. 2002), pemI-pemK Zhang et al. 2004) and ccdA-ccdB (Loris et al. 1999) modules, and the chromosomally encoded relB-relE (Pedersen, et al. 2003; Takagi, et al. 2005), chpBI-chpBK (Zhang et al. 20055), mazE-mazF (Kamada et al. 2003; Zhang et al. 2003a; Zhang et at 20035) and yefM-YoeB (Christensen et al. 2004; Kamada et al 2005) modules from the E. coli genome. In addition, the E. coli genome contains two more TA modules of unknown function, dinf-yafQ and hipB-hipA. The hipB-hipA module has been implicated to play a role in persistence leading to multi-drug resistance (Keren et al. 2004; Korch et al. 2003). Interestingly, all TA operons appear to enlist similar modes of regulation, autoregulation by the antitoxins and their complexes with toxins. Furthermore, (p)ppGpp which is known to be produced under various stresses appears to play an important role in induction of the TA operons (see review by Gerdes et al., 2005). One of these toxins, CcdB directly interacts with gyrase A and blocks DNA replication (Bahassi et al., 1999, Kampranis et al., 1999). Kid has been proposed to interact with DnaB, the helicase required for chromosomal replication and cell growth (Ruiz-Echevarria et al., 1995). RelE appears to act as a ribosome-associating factor that promotes mRNA cleavage at the ribosome A site (Hayes and Sauer, 2003). PemK (Zhang et al., 2004) and MazF (Zhang et al., 2003b) target free mRNA for degradation.

Recent emergence of multi-drug resistant bacteria is a major threat to public health. In particular, the recent finding of vancomycin-resistant bacteria has been a serious concern, since vancomycin is considered to be the last resort against multi-drug resistant pathogens. Therefore, development of new antibiotics is urgently needed, especially the one that targets novel cellular functions, which have not been exploited previously as targets for conventional antibiotics currently available.

As bacterial pathogens can be used in bioterrorism, it is crucial to develop potent non-conventional antibiotics targeting novel cellular function such as bacterial suicide TA systems.

### SUMMARY OF THE INVENTION

The invention provides method for identifying an agent which prevents or partially prevents an antitoxin from forming a complex with its cognate toxin, comprising contacting a potential agent with a labeled substrate in solution, whereby detection of the label indicates presence of an agent that prevents an antitoxin from forming complex with a toxin. The invention also provides an agent capable of interfering with formation of a toxin-antitoxin complex.

### BRIEF DESCRIPTION OF THE DRAWINGS

FIG. 1. Regulation of the mazE-mazF operon. MazE and MazF mRNAs are synthesized from the same operon. One MazE dimer can bind to two MazF dimers to inhibit MazF endoribonuclease activity and the resulting heterohexamers negatively autoregulate the TA operon MazE dimers are subject to cleavage by ClpPA and can also autoregulate the TA operon transcription, but much more weakly than the MazE-MazF heterohexamers complex. MazF dimers, when not bound by MazE, function as MIase to cleave mRNAs specifically at ACA sequences (Zhang et al., 2003b). This MazF endoribonuclease activity leads to bacterial cell growth arrest and eventual cell death. All the other TA systems appear to be also negatively autoregulated in a similar manner.

FIG. 2. X-ray structures of toxin-antitoxin complexes. A. The MazF-MazE complex. One MazE (cyan if in color/pale gray on right) is bound to two MazF honiodimer (blue and light blue if in color/dark gray and extra pale gray) (Kamada et al., 2003). B. The RelE-RelB complex. Two RelB monomers (yellow and light blue if in color/palest gray on left and extra pale gray on right) bind to the RelE dimer (green and blue if in color/gray on left and dark gray on right). When bound to ROE, RelB exists as a monomer with an extended conformation (Takagi et al., 2005). C. The YoeB-YefM heterohexamer complex. Each of two YefM monomers (blue/light blue and cyan/green if in color/dark gray/extra pale gray towards bottom and pale gray/gray towards top) forms a heterotrimeric complex with a single YoeB monomer (light green and orange if in color/light gray upper left and medium gray toward lower right-hand side) (Kamada and Hanaoka, 2005).

FIG. 3. X-ray structures of various toxin-antitoxin complexes [modified from Buts et al. (2005) Trends in Biochern. Sci. 30, 672-679].

(a) The MazF-MazE (4:2) heterohexameric complex. When bound to MazF (gray-white surface), MazE consists of a globular dimerization domain (light blue and pink if in color/pale gray and paler gray) flanked by two C-terminal MazF recognition domains with an extended conformation (dark blue and red if in color/dark gray on left and gray on right). In the absence of MazF, the C-terminal domain of MazE is not ordered (Kamada et al., 2003).

(b) The YoeB-YefM (1:2) heterotrimeric complex. Two YefM monomers form a heterotrimeric complex with a single YoeB monomer. In one YefM monomer, the N-terminal domain is fully ordered (dark blue if in color/dark gray on left) and binds to YoeB (gray-white surface representation), inducing a conformational change in the catalytic site. The corresponding part of the second YefM monomer (red if in color/gray in middle if not in color) is only partially ordered in the absence of a second bound YoeB monomer (Kamada and Hanaoka, 2005).

(c) The RelE-RelB (2:2) heterotetrameric complex. When bound to RelE, RelB exists as a monomer with an extended conformation. In the absence of its toxin partner, it is assumed to be unfolded. Two RelB monomers (red and blue if in color/dark gray (blue) on left and gray (red) on right) bind to the RelE dimer (gray surface) (Takagi et al., 2005).

FIG. 4. Structures of the fluorescent probe and the quencher. A. The structure of ROX, 6 carboxyl-X-rhodamine. B. The structure of the Eclipse quencher. This compound is a non-fluorescent molecule that quenches fluorescence over a broad wavelength range from 400 to 650 nm.

FIG. 5. Assay of MazF activity using CBS-1.

A. Cleavage of CBS-1. The reaction was carried out as described in the text. Fluorescence was measured at 635 nm with excitation at 550 nm. The amounts of MazF used are shown at the left hand side of the figure in pmoles. B. The rate of the MIase reaction against time on the basis of the data from A.

FIG. 6. Coexpression of toxins and antitoxins with the use of a T7 expression system in strain BL21(DE3). Cell cultures grown to log phase were incubated in the presence of 1 mM IPTG for 4-5 h at 37° C. Total cellular proteins were subjected to sodium dodecyl sulfate (SDS)-polyacrylamide gel electrophoresis, followed by Coomassie Brilliant Blue staining. M, protein marker; lane 1, in the absence of IPTG; lane 2, BL21(DE3)/pET21phd-doc; lane 3, BL21(DE3)/pET21hipB-hipA; lane 4, BL21 (DE3)/pET21 dinJ-yafQ; lane 5, BL21(DE3)/pET21mazE-mazF; lane 6, BL21(DE3)/pET21yefM-yoeB; lane 7, BL21(DE3)/pET28higB-higA; lane 8; BL21(DE3)/pET21chpB-chpBK; lane 9, BL21(DE3)/pET21vapB-vapC, and lane 10, BL21(DE3)/pET21relB-relE, For all operons, the 3′-end gene products such as Doc, HipA, YafQ, MazF, YoeB, HigB, ChpBK, VapC and RelE were His-tagged at their C-terminal ends except for HigB which has His tag fused at its N-terminal end. The bands corresponding to the toxins and antitoxins are indicated with green triangles and red circles, respectively. Note that MazF and His-MazF (lane 5) co-migrated at the same position under this condition.

FIG. 7. Expression of YafQ, but not YoeB or RelE, in yeast cells results in cell death or growth arrest.

Equivalent amounts of wild type yeast cells containing the 2-μm expression plasmid pYES2 (that enables the induction of toxin expression using galactose) were spotted onto SC-ura plates to maintain selection of the expression plasmid; cells were serially diluted (1:2) from left to right.

FIG. 8. YoeB expression inhibits new protein synthesis in vivo and in vitro. Panel A, incorporation of [³⁵S]Met into exponentially growing E. coli cells with and without YoeB induction. Equivalent amounts of cell lysate, derived from equal culture volumes, were subjected to SDS-PAGE followed by autoradiography. Panel B, in vitro translation using an E. coli extract (Promega) plus increasing amounts of recombinant YoeB. Positions of molecular weight markers are shown in the center lane: 216, 132, 78, 45.7, 32.5. 18.4 and 7.6 kDa.

FIG. 9, YoeB degrades mRNA with distinctly different kinetics than MazF.

lpp (major outer membrane lipoprotein) mRNA stability was followed by Northern analysis after induction of either YoeB from M. tuberculosis (MTb; top panel) or E. coli (middle panel) or E. coli MazF (bottom panel).

FIG. 10. Interaction of YoeB with the 70S ribosome shifts the position of the ribosome on an mRNA template.

Toeprinting assay to measure the effect of YoeB on a translation initiation complex. A 140 nt 5′ mRNA fragment from mazG was created by T7 RNA polymerase and used to assemble 70S ribosomes and/or other components of the initiation complex as shown. The positions of the relevant products are indicated to the left. “Ribosome” refers to 70S ribosomes, “tRNA” refers to tRNA^{fMet}. A DNA sequencing ladder of the corresponding fragment of mazG was used to determine the sequences where the primer stopped extending and estimate the distance between products.

FIG. 11. YoeB associates with the large 50S ribosomal subunits.

Ribosome fractions were harvested from cells at exponential phase, with or without arabinose mediated YoeB expression (10 min), and separated by centrifugation over a sucrose density gradient. Bottom panel reflects the amount of YoeB protein detected in representative fractions in the profile directly above it, by Western Blot analysis. The high peak on the right represents tRNAs and soluble proteins that sediment at the top of the sucrose gradient.

FIG. 12. In vivo primer extension experiments with ompA and ompF mRNAs. After 2-h induction of YoeB in the presence of arabinose, total RNA was extracted for the primer extension experiments. As shown, primer extension was blocked 3 bases for ompA and 6 bases for ompF mRNAs downstream of the initiation codon. No other bands were observed. The initiation codons (GTA) and the Shine-Dalgarno sequences (GGAG) are shown in gray (if in color, initiation codons are red, Shine-Dalgarno are blue).

FIG. 13. Northern blot analysis after Doc induction. The doe gene was induced with use of a pBAD vector by the addition of arabinose. At the times after induction indicated on top of the gels, total cellular RNAs were extracted and analyzed by Northern blot for ompA, tufA and ompF mRNAs.

FIG. 14. Polysome patterns of cells without (left panels) or with Doc induction (right panels) using cells harboring pBADdoc. Polysome patterns were analyzed as described in FIG. 11 with and without Doc induction by the addition of arabinose. Polysome patterns were analyzed in the presence (upper panels) or in the absence of hygromycin, an antibiotic that blocks translation elongation reaction.

FIG. 15. YafQ exhibits site-specific endoribonuclease activity in vivo.

In vivo primer extension analysis of a portion of the era gene revealed enhanced cleavage by YafQ (YafQ induction time points are the 5 min through 120 min lanes under the red line relative to wild type E. coli BW25113 cells containing the era plasmid but not the YafQ plasmid (0, 90, 120 min lanes flanking YafQ samples). Times represent min of YafQ induction in pBAD using 0.2% arabinose. era mRNA was induced with IPTG, 30 min before YafQ induction. The slowest moving band on the left represents the full length primer extension product, the other three bands represent premature termination due to secondary structure in the era mRNA. Bona fide YafQ recognition sites are represented as those cleavage products that increase with time relative to the control. Additional YafQ cleavage sites are noted higher up on the gel but will require the use of a different era primers in order to determine cleavage sites. Apparent cleavage site for YafQ appears to be ACA (complement of that shown on sequencing ladder).

FIG. 16. DinJ forms a stable complex with YafQ.

The dinJ-yafQ module was cloned into a pET expression vector to enable the addition of a His₆ tag to only the carboxy terminus of YafQ. Samples in the left and right panels were induced for the times shown, subjected to SOS-PAGE and stained with Coomassie blue. Upon affinity chromatography of the samples from the left panel, the panel on the right demonstrates that DinJ copurifies with YafQ. The purified DinJ-YafQ bands are currently being verified by MALTI-TOF mass spectroscopy.

FIG. 17. Sequence alignments of MazF homologues from B. subtilis, B. anthracis, and S. aureus with E. coli MazF.

Identical residues are in black background, and homologous residues in gray background

FIG. 18. Phylogenetic relationships of 23 M. tuberculosis VapC (mt-1 to mt-23). VapC from Dichelobacter nodosus, Leptospira interrogans and Salmonella dublin are also included together with putative other M. tuberculosis toxins, MazJ(mt-1) and MazJ(mt-2).

FIG. 19. Cloning the cycle GFP (ΔNdeI) gene. The GFP fragments will be amplified by PCR using either 5′ ATCACATATGATGGCCAGC AAAGGAGAA 3′ and 5′ AATACGAATTCGCTTTTGTAGAGCTCGTC 3 or 5′CATGAATTCATG GCCAGCAAAGGAGAA 3′ and 5′ AATAGCGGCCGCTTAGCTTTTGTAGAGCTCGTC 3′ using pGFP(ΔNdeI) plasmid (sequences underlined correspond to the recognition sites of restriction enzymes).

FIG. 20. Schematic maps of pET21-GFP/His and pET28-His/GFP plasmids.

(A) EcoRI and NotI and (B) NdeI and EcoRI will be used for cloning of target genes. Restriction enzymes shown with asterisks are not unique sites.

FIG. 21. Interaction between Ni-NTA and a His-tagged TA complex. The magnetic beads are pulled to the bottom of the tube when transferred the released GFP-tagged protein to measure its fluorescence.

### DETAILED DESCRIPTION OF THE INVENTION

One embodiment of this invention is a method to screen for agents which interfere with an antitoxin such that it cannot form complex with its cognate toxin. Such agents may act as antibiotics to inhibit bacterial growth. Different from conventional antibiotics, the antibiotics targeting the toxin-antitoxin (“TA”) complex formation are expected to cause a synergistic inhibitory effect on cell growth by primarily freeing a toxin from the TA complex, which consequently leads to derepress the TA operon expression. As a result, more active toxins are released in the cytoplasm, resulting in more effective growth inhibition and eventual cell death. This is due to the fact that the TA complexes inhibit transcription of TA operons more efficiently than the antitoxins alone.

Almost all bacteria contain toxins that form stable TA complexes with their congate antitoxin in the cells so that toxins are not able to exert their toxic effects on the cells. The invention provides high throughput screening for small chemicals that are able to dissociate the TA complexes to release toxins in the cells. This screening technique in turn facilitates the detection of a novel class of antibiotics, also encompassed by this invention.

Embodiments of the present invention encompass screening systems for agents disruptive of any TA system, including TA systems whose toxins function as any mRNA interferase (MIase). According to the invention, specific cleavable beacon substrates are synthesized for each MIase according to the method described above. Screening systems specific for individual TA systems whose toxins function as MIases are therefore provided herein. Other embodiments of the present invention encompass screening systems for non-MIase toxins using GFP-fusion TA complexes with His-tags for separation as described below.

Accordingly, the invention provides a method for identifying an agent which prevents or partially prevents an antitoxin from forming a complex with its cognate toxin. The agents of this invention preferably interfere with antitoxins such that they cannot form complexes with their cognate toxins. By targeting formation of such complexes, the agents of this invention are valuable as novel, non-conventional forms of antibiotics.

The agents of this invention include those that specifically target certain bacteria or certain groups of bacteria. Accordingly, the screening (identification) methods of the invention are extremely sensitive, i.e., specific, to each particular TA system.

The agent may be any molecule and is preferably a small molecule or chemical, but the invention is not limited to small molecules. Large molecules that may be covered by the invention include peptides, polypeptides, and proteins, among others.

The methods of this invention comprise contacting a potential agent with a labeled substrate in solution. If used to identify agents functioning as mRNA interferases, in one embodiment, the substrate may comprise a short DNA-RNA chimeric substrate. Such substrates are ideally about 5 to about 20 nucleotide bases in length, more preferably about 12 nucleotide bases. The labeled substrate may be a cleavable beacon substrate specific for a particular or more than one particular TA system. Typically, an MIase inhibitor cleaves a certain key base, i.e., rU residue riboneclotide to be cleaved by a MazF toxin. Therefore, one embodiment of the cleavable substrate uses a modified substrate comprising a cleavable site between rU and dA. The potential agent, if acting as a MazF or other toxin, would cleave at that site. The probes useful in this invention are fluorescent at the 5′ end with a quencher at the 3′ end. In preferred methods, the fluorescent probe is ROX, and the quencher is Eclipse. When cleaved, the fluorescent probe is detached from the quencher and fluoresces. Such probes or substrates are called Cleavable Beacon Substrates (CBS). Other probes known in the art may be used with the methods of the invention. Detection of the labeled probe (when cleaved) indicates presence of an agent that prevents an antitoxin from forming a complex with a toxin.

In one embodiment, the substrate is dGdAdTdArUdAdCdAdTdAdTdG. In another embodiment, the substrate is cleavable beacon substrate (CBS-1) and is used to identify agents which prevent MazE/MazF complex formation.

In another embodiment, the substrate is dGdAdTdArUrArCdGdTdAdTdG. In another embodiment, the substrate is cleavable beacon substrate (CBS-2) and is used to identify agents which prevent ChpBI/ChpBK complex formation or YdcD/YdcE complex formation.

In another embodiment, the substrate is dGdAdTdArUrArCdCdTdAdTdG. In another embodiment, the substrate is a cleavable beacon substrate (CBS-3) and is used to identify agents which prevent YdcD/YdcE complex formation.

In another embodiment of the method of the invention useful for non-MIase type toxins, the substrate comprises a Green Fluorescent Protein (GFP)-tagged antitoxin and His-tagged toxin. Alternatively, the substrate comprises a His-tagged antitoxin and GFP-tagged toxin. The GFP-tagged toxin or GFP-tagged antitoxin contain a linker situated between the GFP and the toxin or between the GFP and the antitoxin. The linkers of the invention are of varying lengths, depending the protein, to provide optional function of the protein. The GFP fusion should not inhibit TA complex formation. The appropriate sized linker may readily be determined for each GFP-fusion TA complex.

The dissociation of the substrate, i.e., TA complexes, by an agent is detected by measuring GFP fluorescent signals generated from GFP-tagged antitoxins in solution after removing His-tagged toxins using Ni-NTA Magnetic Agarose Beads. Alternatively, if the GFP-tag is fused to the toxin instead of the antitoxin, and the His-tag is attached to the antitoxin instead of the toxin, dissociation of TA complexes is detected by measuring GFP fluorescent signals generated from GFP-tagged toxins in solution after removing His-tagged anti-toxins using Ni-NTA Magnetic Agarose Beads.

The invention further provides an agent identified by any of the methods of the invention. Thus, the agents of the invention are capable of interfering with formation of a TA complex, and act as non-conventional antibiotics. The TA complex is typically from a bacterial cell. The novel antibiotics of the invention are preferably directed against human pathogenic bacteria.

The invention also provides a composition comprising one or more different agents of the invention in combination with one or more different conventional antibiotics. This composition may be a pharmaceutical composition additionally comprising pharmaceutical excipients.

More than one agent optionally used in combination with one or more conventional antibiotic will provide an additive or synergistic effect of such agents and/or antibiotics. Such different agents may affect more than one TA complex (system) in one pathogenic bacteria, either partially or entirely inhibiting the TA complex.

Further, the invention provides a method for killing or inhibiting growth of microbial cells comprising contacting the pathogens with an agent of invention. Further, the invention provides a method of treating an infection comprising administering any of the pharmaceutical compositions of the invention. Such infections may be tuberculosis, antibiotic-resistant or multi-drug resistant bacteria, such as bacteria resistant to vancomycin, for example. The methods of the invention also cover pathogens used for bioterrorism.

Also provided is a method of regulating bacterial cell dormancy is regulated by contacting the cell with an agent of the invention to cause the cell to become dormant instead of causing the cell to die.

“Pathogen” “microbial agent” “infective agent” are all used interchangeably herein to mean a biological agent that causes disease or illness to its host. An “infection” as used herein is the entry of a host organism by a foreign species.

The compositions of the invention may be administered orally, buccally, parenterally, intranasally, rectally, or topically. Pharmaceutical carriers and excipients used in the methods of the invention are those known in the art.

The term “inhibitor” refers to an agent that prevents, reduces, blocks, neutralizes or counteracts the effects of another agent.

The term “cDNA” refers to a single stranded complementary or copy DNA synthesized from an mRNA template using the enzyme reverse transcriptase. The single-stranded cDNA often is used as a probe to identify complementary sequences in DNA fragments or genes of interest.

As used herein, the terms “encode”, “encoding” or “encoded”, with respect to a specified nucleic acid, refers to information stored in a nucleic acid for translation into a specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code.

One of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. The term “conservatively modified variants” applies to both amino acid and nucleic acid sequences. With respect to particular nucleic acid sequences, conservatively modified variants refers to those nucleic acids which encode identical or conservatively modified variants of the amino acid sequences. Because of the degeneracy of the genetic code, a large number of functionally identical nucleic acids encode any given protein. For instance, the codons UUA, UUG, CUU, CUC, CUA, and CUG all encode the amino acid leucine. Thus, at every position where a leucine is specified by a codon, the codon can be altered to any of the corresponding codons described without altering the encoded polypeptide. Such nucleic acid variations are “silent variations” and represent one species of conservatively modified variation. Every nucleic acid sequence herein which encodes a polypeptide also, by reference to the genetic code, describes every possible silent variation of the nucleic acid. One of ordinary skill will recognize that each codon in a nucleic acid (except AUG, which is ordinarily the only codon for methionine, and UGG, which is ordinarily the only codon for tryptophan) can be modified to yield a functionally identical molecule. Accordingly, each silent variation of a nucleic acid which encodes a polypeptide of the present invention is within the scope of the present invention.

The present invention includes active portions, fragments, derivatives, mutants, and functional variants of mRNA interferase polypeptides to the extent such active portions, fragments, derivatives, and functional variants retain any of the biological properties of the mRNA interferase. An “active portion” of an mRNA interferase polypeptide means a peptide that is shorter than the full length polypeptide, but which retains measurable biological activity. A “fragment” of an mRNA interferase means a stretch of amino acid residues of at least five to seven contiguous amino acids, often at least about seven to nine contiguous amino acids, typically at least about nine to thirteen contiguous amino acids and, most preferably, at least about twenty to thirty or more contiguous amino acids. A “derivative” of an mRNA interferase or a fragment thereof means a polypeptide modified by varying the amino acid sequence of the protein, e.g., by manipulating the nucleic acid encoding the protein or by altering the protein itself. Such derivatives of the natural amino acid sequence may involve insertion, addition, deletion, or substitution of one or more amino acids, and may or may not alter the essential activity of the original mRNA interferase.

The term “gene” refers to an ordered sequence of nucleotides located in a particular position on a segment of DNA that encodes a specific functional product (i.e, a protein or RNA molecule). It can include regions preceding and following the coding DNA as well as introns between the exons.

The term “induce” or inducible” refers to a gene or gene product whose transcription or synthesis is increased by exposure of the cells to an inducer or to a condition.

The terms “inducer” or “inducing agent” refer to a low molecular weight compound or a physical agent that associates with a repressor protein to produce a complex that no longer can bind to the operator.

The terms “introduced”, “transfection”, “transformation”, “transduction” in the context of inserting a nucleic acid into a cell, include reference to the incorporation of a nucleic acid into a prokaryotic cell or eukaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The term “isolated” refers to material, such as a nucleic acid or a protein, which is substantially free from components that normally accompany or interact with it as found in its naturally occurring environment. The isolated material optionally comprises material not found with the material in its natural environment; or, if the material is in its natural environment, the material has been synthetically (non-naturally) altered by deliberate human intervention. For example, an “isolated nucleic acid” may comprise a DNA molecule inserted into a vector, such as a plasmid or virus vector, or integrated into the genomic DNA of a prokaryotic or eukaryotic cell or host organism. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it is generally associated in its natural state (i.e., in cells or tissues). An isolated nucleic acid (either DNA or RNA) may further represent a molecule produced directly by biological or synthetic means and separated from other components present during its production.

The term “MazE” as used herein refers to the general class of antitoxins that antagonize the endoribonuclease activity of MazF and active fragments and derivatives thereof having structural and sequence homology thereto consistent with the role of MazF polypeptides in the present invention.

The term “MazF” as used herein refers to the general class of endoribonucleases, to the particular enzyme bearing the particular name and active fragments and derivatives thereof having structural and sequence homology thereto consistent with the role of MazF polypeptides in the present invention.

The family of enzymes encompassed by the present invention is referred to as “mRNA interferases”. It is intended that the invention extend to molecules having structural and functional similarity consistent with the role of this family of enzymes in the present invention.

As used herein, the term “nucleic acid” or “nucleic acid molecule” includes any DNA or RNA molecule, either single or double stranded, and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. Unless otherwise limited, the term encompasses known analogues.

(The term “operator” refers to the region of DNA that is upstream (5′) from a gene(s) and to which one or more regulatory proteins (repressor or activator) bind to control the expression of the gene(s).

As used herein, the term “operon” refers to a functionally integrated genetic unit for the control of gene expression. It consists of one or more genes that encode one or more polypeptide(s) and the adjacent site (promoter and operator) that controls their expression by regulating the transcription of the structural genes. The term “expression operon” refers to a nucleic acid segment that may possess transcriptional and translational control sequences, such as promoters, enhancers, translational start signals, polyadenylation signals, terminators, and the like, and which facilitate the expression of a polypeptide coding sequence in a host cell or organism.

The phrase “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The abbreviation “PCR” refers to polymerase chain reaction, which is a technique for amplifying the quantity of DNA, thus making the DNA easier to isolate, clone and sequence. See, e.g., U.S. Pat. Nos. 5,656,493, 5,33,675, 5,234,824, and 5,187,083, each of which is incorporated herein by reference.

As used herein the term “promoter” includes reference to a region of DNA upstream (5′) from the start of transcription and involved in recognition and binding of RNA polymerase and other proteins to initiate transcription. The term “inducible promoter” refers to the activation of a promoter in response to either the presence of a particular compound, i.e., the inducer or inducing agent, or to a defined external condition, e.g., elevated temperature.

The term “regulate” as used herein refers to the act of inhibiting, promoting, controlling, managing, directing, or adjusting by some standard or principle or the state of being inhibited, promoted, controlled, managed, directed, or adjusted.

The term “repressor” includes a protein or agent that binds to a specific DNA sequence (the operator) upstream from the transcription initiation site of a gene or operon that can regulate a gene by turning it on and off.

The term “ribosomal RNA” (rRNA) refers to the central component of the ribosome, the protein manufacturing machinery of all living cells. These machines self-assemble into two complex folded structures (the large and the small subunits) in the presence of a plurality of ribosomal proteins. In bacteria, Archaea, mitochondria, and chloroplasts, a small ribosomal subunit contains the 16S rRNA, where the S in 16S represents Svedberg units; the large ribosomal subunit contains two rRNA species (the 5S and 23S rRNAs). Bacterial 16S, 23S, and 5S rRNA genes are typically organized as a co-transcribed operon. There may be one or more copies of the operon dispersed in the genome. Eucaryotic cells generally have many copies of the rRNA genes organized in tandem repeats. The 18S rRNA in most eukaryotes is in the small ribosomal subunit, and the large subunit contains three rRNA species (the 5S, 5.8S and 25S/28S rRNAs).

The term “total RNA” includes messenger RNA (“mRNA”, the RNA that carries information from DNA to the ribosome sites of protein synthesis in the cell where it is translated into protein), transfer RNA (“tRNA”, a small RNA chain that transfer a specific amino acid to a growing polypeptide chain during protein translation; ribosomal RNA (“rRNA”), and noncoding RNA (also known as RNA genes or small RNA, meaning genes that encode RNA that is not translated into protein).

The term “sodium dodecyl sulfate-polyacrylamide gel electrophoresis” is abbreviated SDS-PAGE.

The terms “variants”, “mutants” and “derivatives” of particular sequences of nucleic acids refer to nucleic acid sequences that are closely related to a particular sequence but which may possess, either naturally or by design, changes in sequence or structure. By “closely related”, it is meant that at least about 60%, but often, more than 85%, of the nucleotides of the sequence match over the defined length of the nucleic acid sequence. Changes or differences in nucleotide sequence between closely related nucleic acid sequences may represent nucleotide changes in the sequence that arise during the course of normal replication or duplication in nature of the particular nucleic acid sequence. Other changes may be specifically designed and introduced into the sequence for specific purposes. Such specific changes may be made in vitro using a variety of mutagenesis techniques. Such sequence variants generated specifically may be referred to as “mutants” or “derivatives” of the original sequence.

A skilled artisan likewise can produce protein variants having single or multiple amino acid substitutions, deletions, additions or replacements. These variants may include inter alia: (a) variants in which one or more amino acid residues are substituted with conservative or non-conservative amino acids; (b) variants in which one or more amino acids are added; (c) variants in which at least one amino acid includes a substituent group; (d) variants in which amino acid residues from one species are substituted for the corresponding residue in another species, either at conserved or non-conserved positions; d (d) variants in which a target protein is fused with another peptide or polypeptide such as a fusion partner, a protein tag or other chemical moiety, that may confer useful properties to the target protein, such as, for example, an epitope for an antibody. The techniques for obtaining such variants, including genetic (suppressions, deletions, mutations, etc.), chemical, and enzymatic techniques are known to the skilled artisan.

As used herein, the terms “vector” and “expression vector” refer to a replicon, i.e., any agent that acts as a carrier or transporter, such as a phage, plasmid, cosmid, bacmid, phage or virus, to which another genetic sequence or element (either DNA or RNA) may be attached so as to bring about the replication of the attached sequence or element and so that sequence or element can be conveyed into a host cell.

It must be noted that as used herein and in the appended claims, the singular forms “a”, “and”, and “the” include plural referents unless the context clearly dictates otherwise. All technical and scientific terms used herein have the same meaning.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

To screen for potential agents that interfere with the TA complex, a number of TA complexes will be used from human pathogens and E. coli, which can be easily expressed and purified using an E. coli expression system. In order to detect the dissociation of the TA complexes, highly sensitive high-throughput methods may be used, which are dependent on fluorescence detection using either beacon-type of RNA substrates for mRNA interferase (MIase) toxins or GFP-fusion TA complexes for non-MIase toxins.

### Toxin-Antitoxin (TA) Systems

### The MazE-MazF Toxin-Antitoxin System

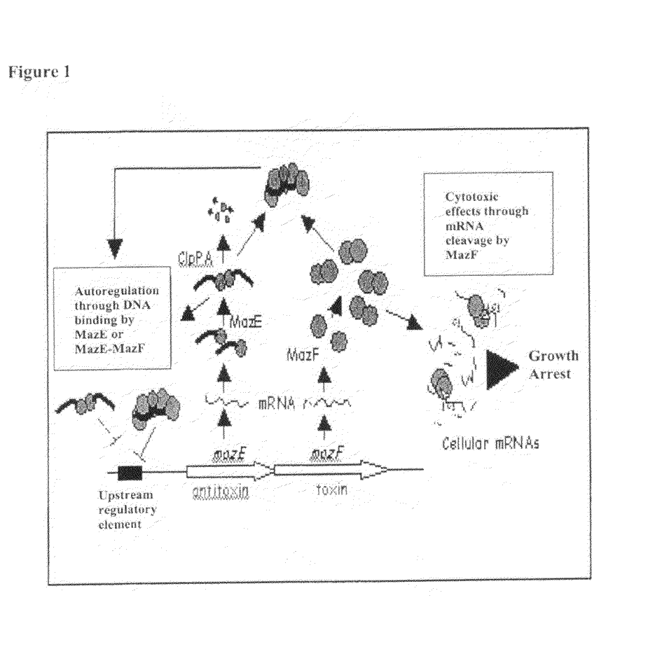

In the MazEF TA system (Aizenman et al., 1996; Kamada et al., 2003; Marianovsky et al., 2001; Zhang et al., 2003b), the MazF toxin is stable and the MazE antitoxin/antidote is labile. The short half-life of MazE is due to degradation by ATP-dependent serine protease, ClpPA (Aizenman et al., 1996). The operon is either negatively autoregulated by MazE or a MazE-MazF complex (Marianovsky et al., 2001; Zhang et al., 2003a). Its regulation by guanosine-3′,5′-bis-pyrophosphate (ppGpp) proposed by Engelberg-Kulka (Aizenman et al., 1996) has been much disputed and it seems likely that ppGpp does not directly regulate the mazEF transcription but indirectly regulates the activation of MazF (for example, through Lon protease) (Gerdes et al., 2005). MazEF-mediated cell growth arrest occurs when transcription of the TA module and/or translation of the mazEF mRNA is inhibited, as MazE is much more unstable than MazF. Thus, MazF is freed from its complex with MazE as depicted in FIG. 1.

Activation of MazF occurs by severe amino acid or thymine starvation (Sat et al., 2003), certain antibiotics such as rifampicin and chloramphenicol (Sat et al., 2001), the toxic protein Doc (Hazan et al., 2001) or other stress conditions such as high temperature, oxidative stress and DNA damage (Hazan et al., 2004).

### MazE and MazF Structure and Function

MazF has been historically categorized as an inhibitor of translation. However, the target of this inhibition is actually mRNA—not the translation apparatus—as we have recently demonstrated that MazF is a sequence-specific endoribonuclease (Zhang et al., 2003b). MazF displays remarkable substrate specificity. It only cleaves single stranded RNA, (not DNA or dsRNA) predominantly between the A and C of the sequence ACA. Cellular tRNAs appear to be protected from cleavage because of their extensive secondary structure, while rRNAs appear to evade degradation by MazF because of their close association with ribosomal proteins. Therefore, MazF expression results in nearly complete degradation of mRNAs, leading to severe reduction of protein synthesis in conjunction with growth arrest (Zhang et al., 2003b). Proteins with sequence similarity to MazF are found in a number of bacteria or on their extrachromosomal plasmids. An 8100 plasmid-encoded toxin in E. coli called PemK is also a sequence-specific endoribonuclease with broader cleavage specificity than that of MazF (Zhang et al., 2004). MazF and its functional counterparts in E. coli and other bacteria as mRNA interferases (MIases).

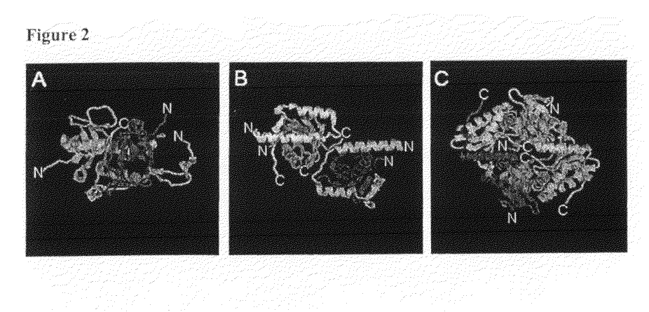

The X-ray structure of the MazE-MazF complex has been solved (Ramada et al., 2003). This, along with the crystal structures of two other individual toxins without their antidote partners (Hargreaves et al., 2002; Loris et al., 1999), revealed that considerable structural similarity exists between all three toxins albeit their different targets and sequences. Consistent with data from biochemical studies indicating that MazF (111 aa) forms a stable complex with MazE (82 aa) at a ratio of one MazE dimer to two MazF dimers (Zhang et al., 2003a), the X-ray crystal structure of the MazE and MazF complex consists of a 2:4 heterohexamer composed of alternating MazE and MazF homodimers (F2-E2-F2, FIG. 2A). Interestingly, the C-terminal region of MazE is highly negatively charged and disordered, and extends over the cleft formed between two MazF molecules in the MazF homodimer. This charged extension on MazE may mimic the structure of single stranded RNA and disrupt the endoribonuclease activity of MazF by blocking its RNA substrate-binding site (Zhang et al., 2003b).

Structural study of TA complexes has greatly increased our understanding of how individual toxins form stable complexes with their cognate antitoxins. In addition to the X-ray structure of the MazE-MazF complex (Kamada et al., 2003) (FIG. 2A), the crystal structures of the RelB-RelE complex (Takagi et al., 2005) (FIG. 2B) and the YefM-YoeB complex (Kamada and Hanaoka, 2005) have been recently determined. In each complex structure, antitoxin interacts with its cognate toxin in a different manner as discussed in more detail below. The NMR structures of the MazF-substrate analogue complex and RelB NMR solution structure have been recently determined.

As shown in FIG. 2, in each TA system, a toxin interacts with its cognate antitoxin in a unique manner, specific to the TA complex. Therefore, highly unique antibiotics may be developed only for a specific pathogenic bacterium or a group of specific pathogenic bacteria. Furthermore, if a pathogen has more than one TA systems, specific antibiotic for each TA system may be developed. This may lead to an additive or synergistic effect of two different antibiotics on the pathogen. In addition, the use of new antibiotics developed in this proposal with conventional antibiotics is expected to be synergistic as they use completely different cellular targets.

A highly sensitive method will be developed for each TA system to screen chemicals which block the TA complex formation or are able to dissociate the TA complex. These methods may be used for high throughput screening (for example, the NIH Molecular Libraries Screening Center established for the NIH Roadmap Initiative).

The following publications, each of which are incorporated in their entirety by reference herein, further describe bacterial toxins, which include a paper on the MazF-induced quasi-dormancy and the single-protein production system in Mol. Cell.

Characterization of the interactions within the mazEF addiction module of Escherichia coli J. Biol. Chem (2003) 278, 32300-32306 (Zhang et al., 2003a)

We demonstrated that the functional MazEF complex is composed of two MazF dimers plus one MazE dimer. This complex was shown to bind to the ma EF operon. MazE was found to directly bind DNA while MazF enhanced the DNA binding activity of MazE. Finally, the binding interface between MazE and MazF was defined by the yeast two-hybrid system. We concluded that MazE is composed of two domains, the N-terminal DNA-binding domain and the C-terminal domain interacting with MazF. These results are consistent with the X-ray structure of the MazE-MazF complex (Kamada et al., 2003).

MazF cleaves cellular mRNAs specifically at ACA to block protein synthesis in Escherichia coli Mol. Cell (2003) 12, 913-923 (Zhang et al., 2003b)

Using a cell-free system, we demonstrated that MazF inhibits protein synthesis but not DNA replication or RNA synthesis. Subsequently, we demonstrated that MazF is a sequence-specific (ACA) endoribonuclease that acts only on single-stranded RNA. MazF works as a ribonuclease independent of ribosomes, and is, therefore, functionally distinct from RelE, another E. coli toxin, which assists mRNA cleavage at the A site on ribosomes (Pedersen et al., 2003). Upon induction, MazF cleaves almost all cellular mRNAs to efficiently block protein synthesis. Purified MazF inhibited protein synthesis in both prokaryotic and eukaryotic cell-free systems. This inhibition was released by MazE, the labile antitoxin against MazF. Thus, MazF functions as a toxic endoribonuclease that interferes with the function of cellular mRNAs by cleaving them at specific sequences leading to rapid cell growth arrest, and we coined the term, “mRNA interferase” (MIase) for this type of endoribonucleases. The role of such endoribonucleases may have broad implication in cell physiology under various growth conditions.

Interference of mRNA function by the sequence-specific endoribonuclease PemK J. Biol. Chem. (2004) 279, 20678-20684 (Zhang et al., 2004)

The pemI-pemK TA system is on plasmid R100 and helps to maintain the plasmid by post-segregational killing in an E. coli population. We demonstrated that PemK is another MIase that cleaves mRNAs, while Peril blocks this activity. PemK cleaves only single-stranded RNA preferentially at the 5′ or 3′ side of the A residue in the “UAX (X is C, A or U)” sequences. Although PemK was previously thought to inhibit DNA replication through DnaB (Ruiz-Echevarria et al., 1995), we now unambiguously showed that PemK is an MIase. The reported inhibition of ColE1 DNA replication can be readily explained by the PemK's MIase activity on RNAII, a primer for ColE1 DNA replication. Furthermore, the growth inhibition of various eukaryotic cells by PemK induction (de la Cueva-Mendez et al. 2003) can also be explained by PemK's MIase activity against cellular mRNAs.

Insights into the mRNA cleavage mechanism by MazF, an mRNA interferase
J. Biol. Chem. (2005) 280, 3143-3150 (Zhang et al., 2005a)

Using RNA-DNA chimeric substrates containing XACA, MazF cleaves the substrates at the 5′-end of the ACA sequence (between X and A), yielding a 2′,3′-cyclic phosphate at one end and a free 5′-OH group at the other. Using these substrates, we demonstrated that the 2′-OH group of residue X is absolutely essential for MazF cleavage, whereas all the other residues may be deoxyriboses.

Single Protein Production in Living Cells Facilitated by an mRNA Interferase
Mol. Cell (2005) 18, 253-261 (Suzuki et al., 2005)

We found that although MazF induction in E. coli completely inhibits cell growth as a result of degradation of almost all cellular mRNAs by MazF, cells are still fully metabolically active. This was demonstrated by exploiting the ACA-specific MIase activity of MazF, We found that concomitant expression of MazF and a target gene engineered to encode an ACA-less mRNA results in sustained and high-level (up to 90%) target expression in virtual absence of background cellular protein synthesis. Virtually, we converted E. coli cells into a bioreactor producing a single protein and thus the system was termed “single-protein production” (SPP) system.

The fact that cells were still able to produce a single protein of interest under complete cell growth arrest indicates that the metabolic capacity of the cell is intact for an extended period of time, so that not only energy metabolism (ATP production), but also biosynthetic functions for amino acids and nucleotides, are fully active in the growth arrested cells. Furthermore, transcriptional and translational machineries are also well maintained and fully functional. Therefore, the cells under the MazF-induced dormancy are under a novel physiological state, which is termed as “quasi-dormancy”. The discovery of the quasi-dormancy opens an exciting avenue for studying new bacterial physiology that may play important roles in bacterial pathogenicity and persistence in multi-drug resistance.

Characterization of ChpBK, an mRNA Interferase from Escherichia coli
J. Biol. Chem. (2005) 280, 26080-26088 (Zhang et al., 2005b)

ChpBK is a toxin encoded by the E. coli genomic chpBIK TA module, consisting of 116 amino acid residues. Its sequence shows 35% identity and 52% similarity to MazF. We found that ChpBK is another MIase cleaving mRNAs at ACY (U, A, or G) in a manner identical to that of MazF.

### Unpublished Preliminary Results

Characterization of dual substrate binding sites in the homodimeric structure of Escherichia coli mRNA interferase MazF. J. Mol. Biol. (Li et al., 2005) In press

In collaboration with Dr. M. Ikura, Professor at Ontario Cancer Institute, the University of Toronto, Canada, we recently determined the NMR structure of the MazF dimer that forms a complex with a substrate analog. We demonstrated that there are dual substrate binding sites on the concave interface of the MazF homodimer, and thus the MazF homodimer is a bidentate endoribonuclease equipped with two identical binding sites for mRNA processing. However, importantly, a single MazE molecule occupying one of the binding sites can affect the conformation of both sites, hence effectively hindering the MazF MIase activity.

Multiple mRNA Interferases in M. Tuberculosis

We demonstrated that M. tuberculosis contains at least seven genes encoding MazF homologues (mt1 to mt7), four of which caused cell growth inhibition when induced in E. coli. We also found that MazF-mt1, -mt3 and -mt6 function as sequence-specific mRNA interferases similar to E. coli MazF, These results suggest that presence of multiple mRNA intereferases may be important in the multi-dimensional dormancy response of this pathogen in human tissues.

Rationale for Experimental Design—All bacteria including pathogenic bacteria contain suicide genes except for obligate intracellular pathogens such as Chlamydia, Mycoplasma and M. leprae (Pandey and Gerdes, 2005). Particularly it is interesting to note that certain free-living bacteria, which grow very slowly, have a large number of the TA systems; for example, M. tuberculosis contains at least 38 TA systems.

Among those pathogenic bacteria which may be used as biological weapons, B. anthracis contains one MazF-MazE TA system and Yersinia pestis contains five various TA systems. The genome sequence of Clostridium botulinum is not available but its close relative, C. tetani contains at least one Phd-Doc TA system. These facts quite compellingly suggest that bacterial TA systems are ideal targets for the development of new antibiotics, which are distinctively different from the currently available conventional antibiotics.

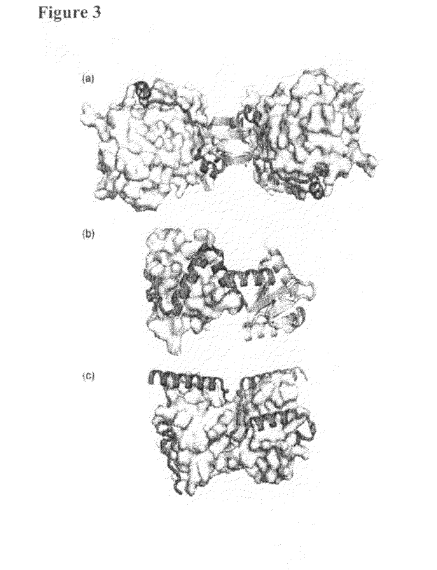

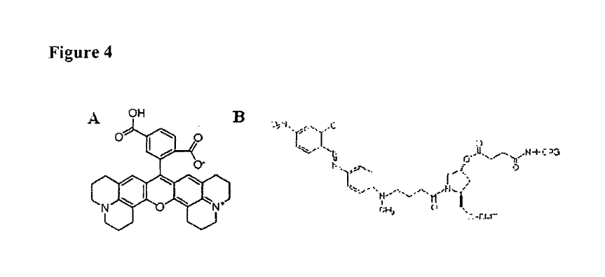

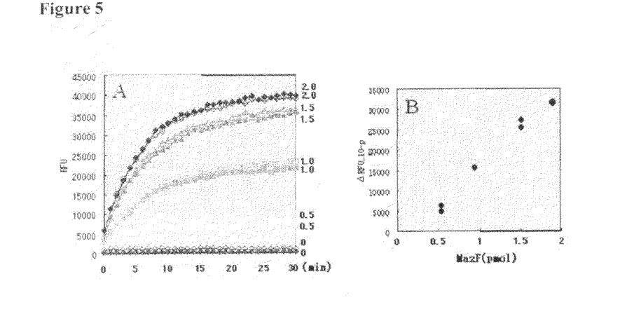

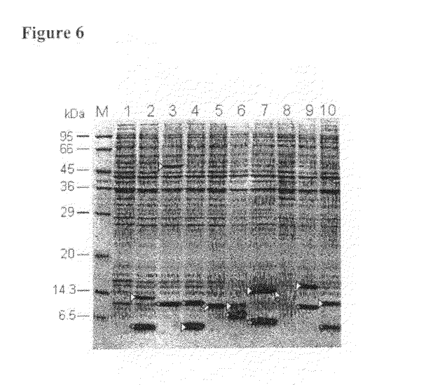

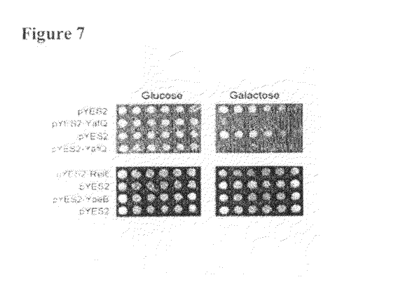

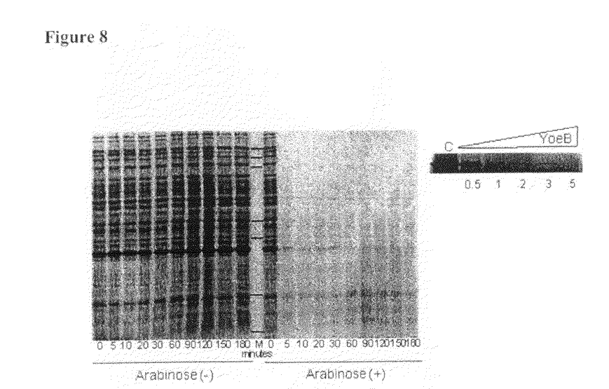

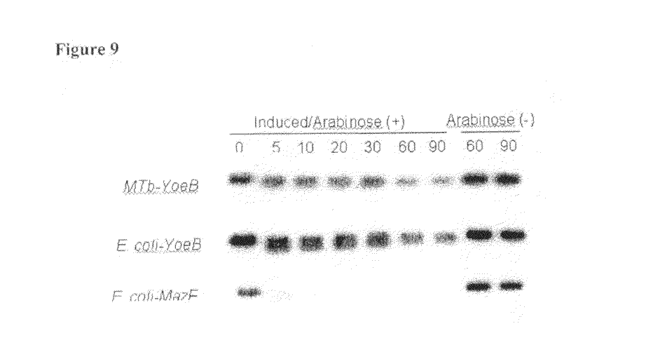

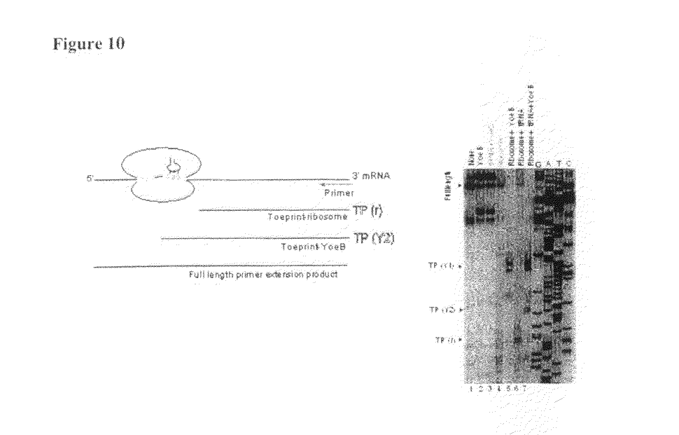

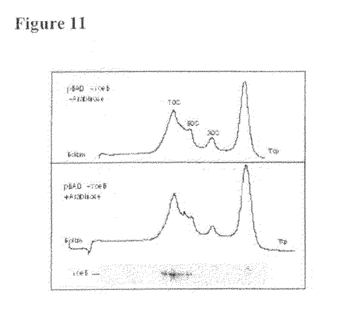

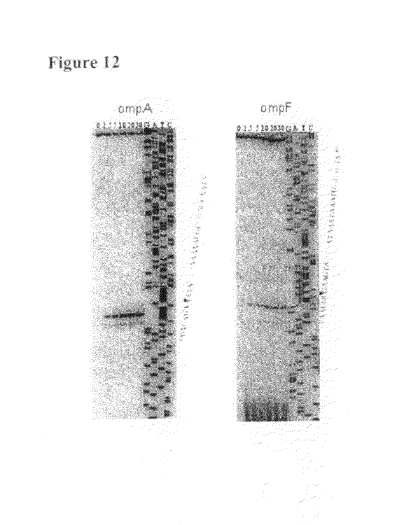

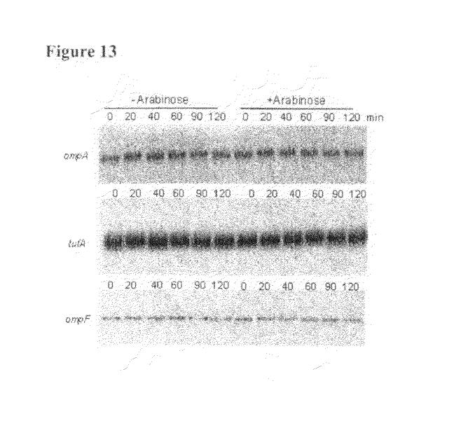

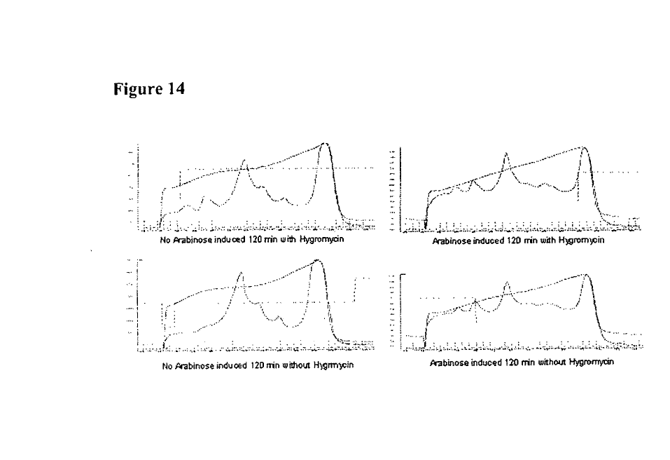

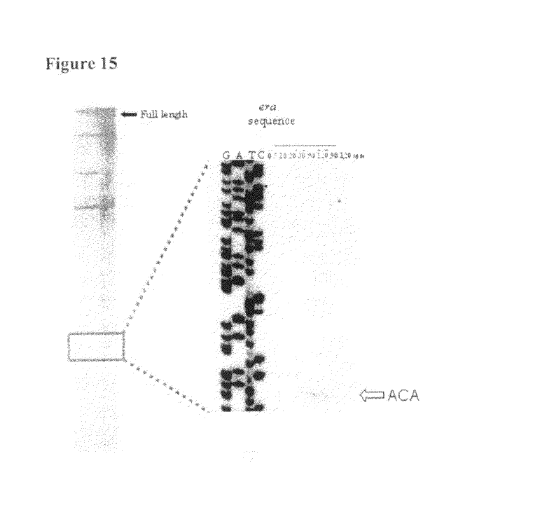

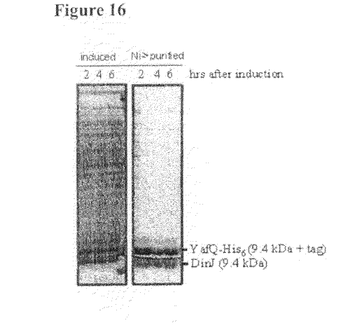

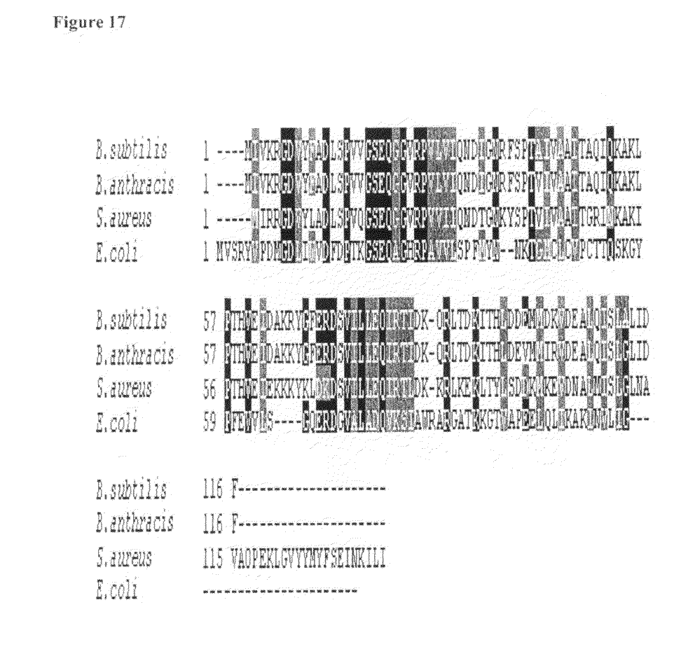

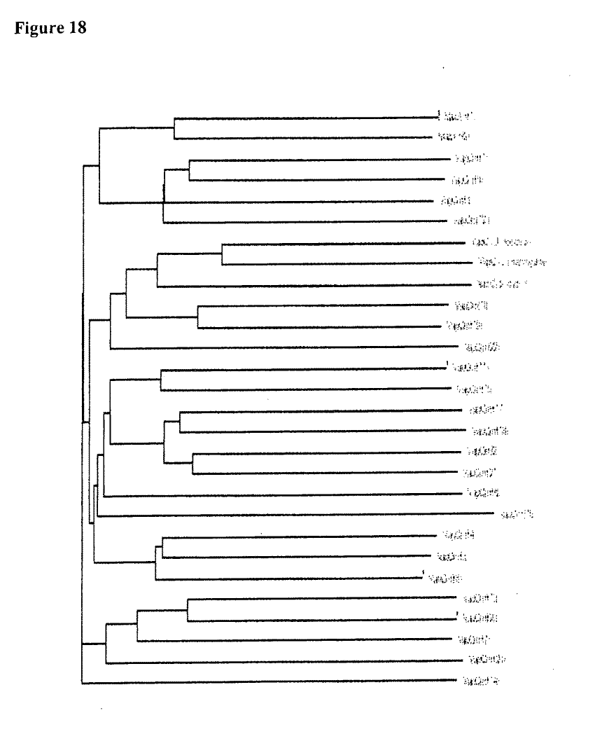

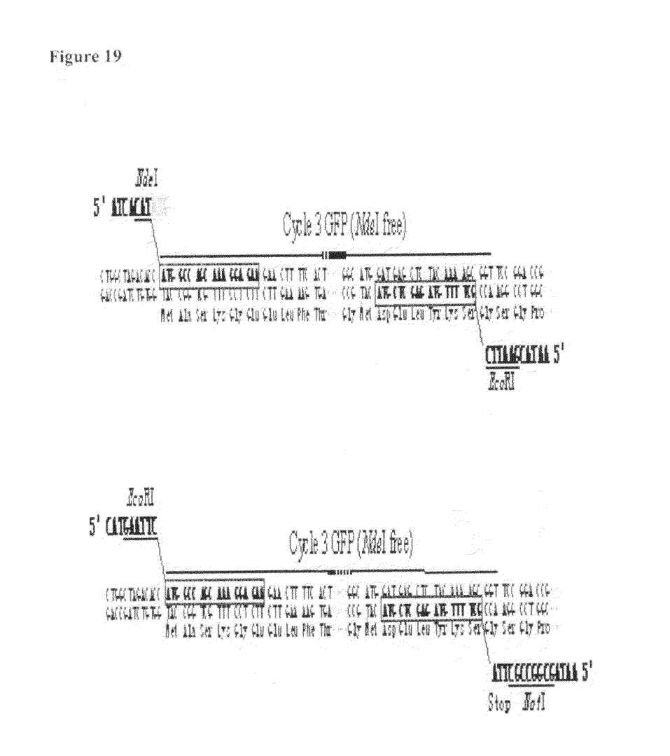

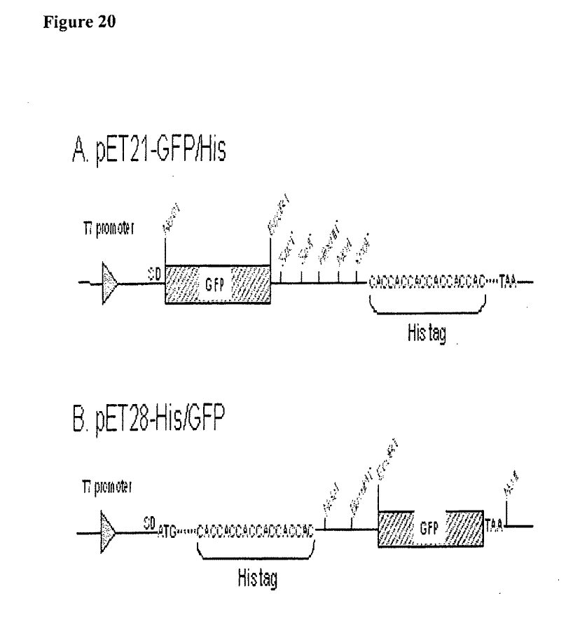

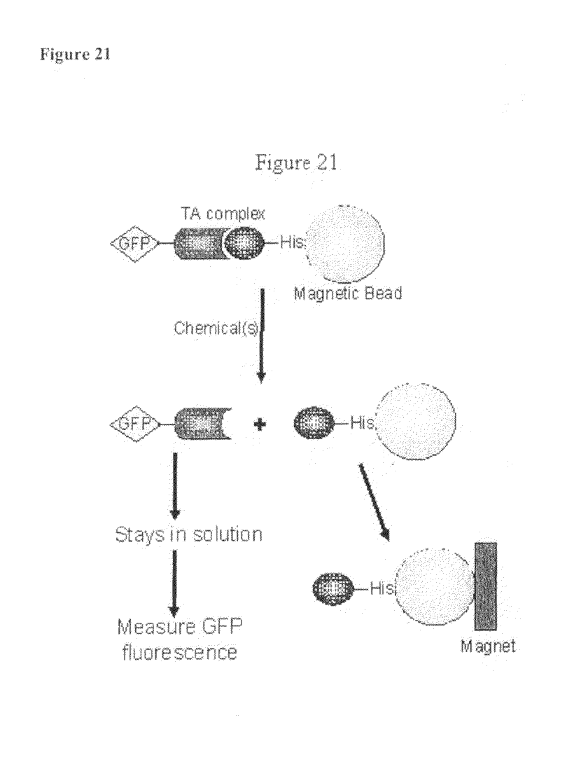

All TA systems are considered to be expressed in the optimally growing cells in the form of the toxin-antitoxin complexes so that toxic effects are suppressed under normal growth conditions. To date, three X-ray structures of the toxin-antitoxin complexes have been solved as shown in FIG. 3. Remarkably, each set forms a unique complex that is different from each other. However, all these antitoxins are much more unstable than their cognate toxins in the cells so that when protein synthesis is blocked under stress conditions, antitoxins are digested by cellular proteases to release toxins in the cells. As a result, cell growth is inhibited which eventually leads to cell death.

Accordingly, any chemical which blocks the interaction between toxins and antitoxins can serve as a potential antibiotic for bacteria for the following reasons: (1) the chemicals will fully or partially release antitoxins from the complexes with their cognate toxins, and the released antitoxins will be quickly removed by cellular proteases resulting in release of free toxins in the cells, (2) the toxin-antitoxin complexes are much stronger repressors for their operons than antitoxins alone, thus, more toxins and antitoxins will be synthesized in the cells in the presence of the chemicals, and (3) the newly synthesized antitoxins will be unable to form the stable complexes with their cognate toxins in the presence of these chemicals. As a result, the cellular concentration of toxins will increase, leading to inhibition of cell growth. The synergistic effect of the antibiotics targeting toxin-antitoxin complexes (the removal of antitoxins induce further production of toxins) is unique and a particularly important feature of the antibiotics of this invention. Another important aspect of this new class of antibiotics is that they may be specific for each toxin-antitoxin complex or only for a group of homologous TA systems, so that it is possible to develop unique antibiotics effective against a specific pathogen.

As we describe below, all the TA complexes from E. coli (MazF-MazE, YoeB-YefM, YafQ-DinJ, RelE-RelB, ChpBK-ChpBI and HipA-HipB) have been isolated and are available in our laboratories, and will be used for development of the individual screening methods. In addition, the YdcE-YdcD complex from B. subtilis (YdcE is 96% identical to the B. anthracis MazF homologue), the HigB-HigA complex from highly virulent E. coli CFT073, the Doc-Phd complex from phage P1 and the VapC-VapB complex from Haemophilus influenzae have been also purified and are readily available in our laboratories. These ten TA complexes encompass almost all known TA systems in bacteria; some of which work as mRNA interferases (MazF, ChpBK and YdcE), while others function as ribosome-associated factors that stimulate ribosomal intrinsic endoribonuclease activity (RelE) or block translation initiation (YoeB). The mechanism of toxicity is yet to be determined for YafQ, HipA, Doc, HigB and VapC. We will also purify the MazF-MazE homologue complexes from S. aureus and B. subtilis and also VapC-VapB complexes from M. tuberculosis. M. tuberculosis contains as many as 23 different VapC-VapB TA systems.

In this invention, methods are provided for detecting the dissociation of toxins from the TA complexes for each TA system upon the addition of agents, which may be small chemicals, other molecules or any agents that partially or totally inhibit TA complex formation. The methods are dependent upon the use of fluorescent probes to detect the released toxins or released antitoxins from the TA complexes upon the addition of small chemicals.

The interaction between toxins and antitoxins occurs in quite extended areas on their surfaces and includes charge and hydrophobic interactions (see FIG. 3). Therefore, a chemical may only partly disrupt the interactions between the two proteins leading to partial inhibition. However, the addition of two or more of these weak inhibitors may result in a dramatic synergistic inhibitory effect, if each one of them interacts with the TA complex at different sites. Accordingly, a new chemical may be designed on the basis of these inhibitory compounds, which will combine their effects. It is also possible to find chemicals that do not directly interfere with the TA interaction, but rather bind to either toxins or antitoxins causing an allosteric conformational change, which results in dissociation of toxins and antitoxins from the TA complexes.

### Study I

### Development of Highly Sensitive Substrates to Detect Sequence-Specific MIase Activities

A well-characterized MIase, MazF will be used as a model protein to develop highly sensitive substrates, with which one can detect even a small amount of MazF released from the MazE-MazF complex in a high-throughput screening of chemicals. For this purpose, we synthesized a short DNA-RNA chimeric substrate of 12 bases, dGdAdTdArU dTdAdTdG. We have shown recently that the rU residue is the key base which has to be a ribonucleotide to be cleaved by MazF (Zhang et al., 2005a). In order to develop the most sensitive method to detect the MazF mRNA interferase activity, we modified this substrate by attaching a fluorescent probe at the 5′ end and a quencher at the 3′ end. This modified substrate is not fluorescent unless it is cleaved between rU and dA, which detaches the fluorescent probe from the quencher. We term this type of substrates for mRNA interferases as Cleavable Beacon Substrates or CBS. We used ROX (6-carboxyl-X-rhodamine) for the 5′-end modification and Eclipse as a quencher at the 3′-end modification (FIGS. 4A and B, respectively). The distance between the two molecules is 12 bases apart, which is sufficient for the Eclipse to quench the fluorescence of the 5′-end ROX. Among a number of fluorescent probes, we chose ROX because it is resistant to photobleaching and is stable over a wide range of pH. We chose Eclipse as a quencher because it is highly stable and therefore can be used safely in all oligonucleotide deprotection reactions. Furthermore, Eclipse is substantially more electron deficient than other quenchers and thus leads to better quenching of a wide range of dyes.

### Methods

Synthesis of a cleavable beacon substrate (CBS) for MazF—The 12-base DNA-RNA chimeric beacon substrate (CBS-1) that emits fluorescence only when it is cleaved (in this case by MazF) was

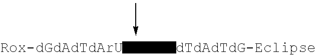

synthesized as follows: Using Epoch Eclipse Quencher CPG (Epoch Biosciences, Inc.) for the 3′-end modification, the DNA-RNA chimeric substrate was synthesized by a DNA/RNA synthesizer (AB13400). For the 5′ end, amino linker (C6) (ABI) was used. For the DNA segments, DNA amidite (Proligo), and for the RNA segment (rU residue), RNA amidite (Proligo) were used for the oligonucleotide synthesis. After synthesis, the oligonucleotide was cleaved off from CPG with use of 28% ammonia (diluted with water):ethanol (3:1). The solution thus obtained was incubated at 55° C. for 6 h to remove the protective groups from each base. After the reaction, the sample was dried with use of a rotary evaporator. The product was then resolved in TEA-3HF/TEA/1-NMP (4:3:6) and the solution was treated at 65° C. for 6 h to remove the protective groups at the 2′-OH group of the rU residue at position 4. [TEA=triethlamine, TEA-3HF=triethylamine-tris-hydrofluoride, and 1-NMP-1-methyl-2-pyrrolidone] After desalting, the product was purified by reverse phase HPLC. The product at this stage is 5′-NH₂-dGdAdTdArUdAdCdAdTdAdTdG-Eclipse-3′. This product was modified with ROX-SE (Invitrogen) at weakly alkaline condition. The reaction mixture was purified by gel filtration to remove free ROX dye. The product thus obtained was further purified with PAGE to separate the ROX-modified product from unmodified products. The final product CBS-1 was freeze-dried after desalting.

Cleavage reaction of CBS-1 by MazF—CBS-1 is expected to be cleaved between rU and dA residues as shown above, resulting in emission of fluorescence. In a pilot experiment, we synthesized a small amount of CBS-1 and performed the cleavage reaction with use of purified MazF. The MIase reaction was carried out as follows; 5 μl (5×) MazF buffer (50 mM Tris-HCl, pH 7.8), 10 μl distilled water and 5 μl of CBS-1 solution (2 pmol/μl) were mixed and the mixture was preincubated at 37° C. The reaction was started by adding different concentrations of MazF (5 μl) (see FIG. 5). The excitation and emission wavelengths used were 550 and 635 nm, respectively. A preliminary result is shown in FIG. 5, from which a number of interesting observations can be made as follows:

1. The 12-base CBS-1 substrate functions as a suitable and sensitive substrate for MazF, indicating that ROX and Eclipse attached at the 5′- and 3′-ends of the 12-base nucleotide, respectively, do not block the MazF MIase enzymatic reaction.
2. There is a linear relationship between the initial rate of the reaction and the MazF concentrations. 3. MazF used in this reaction is fused to the trigger factor (a cold-shock molecular chaperone used for high expression of MazF). Interestingly this fusion protein can be expressed in the absence of MazE, the antitoxin for MazF. The reason for this low toxicity of MazF when fused with the trigger factor is unknown at present. The protein used appears to exhibit only single round cleavage reaction and may not completely cleave the substrate. Nevertheless, the experiment clearly demonstrates that our substrate can detect MIase activity even at low concentrations of MazF and thus will be suitable for use in high throughput screening of potential antibiotics. On the basis of these preliminary results, the experiment will be repeated using purified MazF without any fusion.

Synthesis of specific cleavable beacon substrates for other MIases—To date, in addition to MazF, two more MIases, ChpBK from E. coli K12 (Zhang et al., 2005b) and YdcE from B. subtilis (Pellegrini et al., 2005) have been characterized. We will synthesize the following 12-base CBS substrates for these MIases;

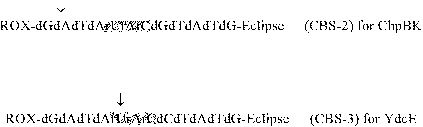

These CBS substrates will be synthesized according to the method described for CBS-1 above. CBS-2 may be cleaved by both ChpBK and YdcE (but not by MazF), while CBS-3 may be cleaved only by YdcE These substrates are important to detect specific MIases and may be used for characterization of unknown MIases whose specificities have not been characterized. As discussed in Study 2, we will design new CBS substrates for YoeB and YafQ after determining their cleavage specificities.

The present invention encompasses screening systems for agents disruptive of any TA system, including TA systems whose toxins function as any MIase. As we find more MIases from Study 2 and determine their specific cleavage sequences, we will synthesize a specific cleavable beacon substrate for each MIase according the method described above. In this way we will be able to develop screening systems specific for individual TA systems whose toxins function as MIases.

### ANTICIPATED PROBLEMS AND THEIR SOLUTIONS

In FIG. 5, the substrate is not being hydrolyzed to completion, as the protein used has single round cleavage activity. As mentioned above, the sensitivity of the reaction will likely improve significantly when the experiment is repeated using purified MazF. However, if the reaction still does not improve, it may mean that this is the intrinsic enzymatic property of MazF or the property of the substrate used. In the latter case, the hydrophobicity of the products (either ROX or Eclipse) may interfere with the second cycle binding of MazF. To counteract this effect, we will incorporate various mild detergents in the reaction that will enhance the accessibility of the substrate by dissociating the bound reaction products without influencing the enzyme activity.

### Study 2

Isolation of Various TA Complexes from E. Coli and Other Pathogenic Bacteria

In this Study, we will isolate a number of TA complexes from non-pathogenic and pathogenic bacteria (see Table 1).

Before proceeding to Study 3 where TA complexes will be used for screening of small chemicals, we will ensure that each TA operon in Table 1 cloned from various bacteria is well expressed in E. coli. This is important for establishing the screening systems.

We will determine the cellular targets for TA systems, which are not yet characterized. This will allow us to develop a unique substrate for each TA system as described for MazF in the previous section.

We have cloned all six TA systems from E. coli K12 (MazF-MazE, ChpBK-ChpBI, RelE-RelB, YoeB-YefM, YafQ-DinJ and HipA-HipB) and expressed these using a T7 expression system as shown in FIG. 6 (also see Table 1). In all cases, TA complexes are well expressed. Since all toxin proteins are His-tagged, all the TA complexes are easily purified by using Ni-NTA resin from which toxins can be further purified as described previously for MazF (Zhang et al., 2003b). Out of these six TA systems, RelE (Hayes and Sauer, 2003; Pedersen et al., 2003), MazF (Zhang et al., 2003b) and ChpBK (Zhang et al., 2005b) have been characterized (see Table 1). We will purify the remaining three toxins, YoeB, YafQ and HipA and identify their cellular targets. In addition, we will isolate the

TABLE 1
List of the TA systems to be studied in this application

| TA module | Bacterium | Length (a.a.) | | Target |
|---|---|---|---|---|
| (Toxin-Antitoxin) | or Phage | Toxin | Antitoxin | of toxin |
| MazF-MazE | E. coli K12 | 111 | 82 | mRNA |
| ChpBK-ChpBI | E. coli K12 | 116 | 83 | mRNA |
| RelE-RelB | E. coli K12 | 95 | 79 | Ribosome |
| YoeB-YefQ | E. coli K12 | 84 | 92 | Ribosome |
| YafQ-DinJ | E. coli K12 | 92 | 86 | mRNA |
| HipA-HipB | E. coli K12 | 440 | 88 | Unknown |
| HigB-HigA | E. coli CFT073 | 90 | 94 | Unknown |
| YdcE-YdcD | B. subtilis | 116 | 93 | mRNA |
| MazFsa-MazEsa | S. aureus | 120 | 56 | mRNA |
| VapC-VapB | H. influenzae | 132 | 78 | Unknown |
| Doc-Phd | Phage P1 | 126 | 73 | Unknown |

HigA-HigB complex from a highly virulent E. coli CFT073 strain. HigA-HigB is one of the most abundant TA systems in bacteria including Y. pestis, the etiologic agent of plague. YdcE-YdcD complex has been reported from B. subtilis. B. anthracis MazF homologue has 93% identity to B. subtilis YdcE, and similarly YdcD has 53% identity to its B. anthracis counterpart. Therefore, all or some of chemicals blocking the YdcE-YdcD complex formation may also inhibit the MazF-MazE homologue complex formation in B. anthracis.

We will also isolate the MazF-MazE homologue from S. aureus, a most common human pathogen that causes a very wide spectrum of diseases ranging from cutaneous infections to life-threatening conditions. Therefore, screening of novel antibiotics against this pathogen is also very important particularly because of emergence of multi-drug resistant strains of this pathogen. We will also isolate a number of VapC-VapB complexes from H. influenzae, a common pathogen in the human respiratory track, and from M. tuberculosis. The latter pathogen contains unusually large number (as many as 23) of the TA systems. This implies that TA system may play an important role the dormancy of this most devastating human pathogen. It should be noted that it is possible to find a chemical, which causes either complete or partial inhibition of the VapC-VapB TA systems in this pathogen. Lastly, we will isolate the Doc-Phd complex from phage P1, whose homologue is found in Vibrio cholerae, another human pathogen.

### Characterization of the Cellular Target of YoeB of E. Coli

Most recently the X-ray structure of the YefM-YoeB (2:1) complex has been determined (Kamada and Hanaoka, 2005). Using purified YoeB, the authors showed that YoeB preferentially cleaves RNA at A or G residues, and speculated that the YoeB toxicity is due to this endoribonuclease activity. The in vitro data presented by Kamada and Hanaoka is consistent with the in vivo data published by Gerdes and his associates (Christensen et al., 2004). However, as shown below, our results clearly indicate that this effect of YoeB is not its primary function. Our preliminary data strongly support the hypothesis that the primary target of YoeB is the translation initiation complex and its specifically inhibits translation initiation. A substantial amount of the preliminary data has been obtained as described below.

However, additional experimentation will unambiguously identify the exact cellular target of YoeB and mechanism of inhibition of translation initiation by this protein.

1. YoeB toxicity is specific to prokaryotes—YoeB is not toxic in yeast in contrast to YafQ, another MIase as described later (FIG. 7). This is consistent with the fact that YoeB binds to 50S ribosomes, which are not conserved between bacteria and yeast.
2. YoeB is a very potent toxin that blocks cell growth and cellular protein synthesis immediately after its induction—Cellular growth (not shown) and protein synthesis is almost completely inhibited within 5 min after YoeB induction using an arabinose-inducible pBAD vector (FIG. 8). In contrast, cellular protein synthesis is inhibited after a longer period (at least 15-20 min) after the induction of MazF (a sequence-specific endoribonuclease whose function is not dependent on the ribosome) (Zhang et al., 2003b).
3. Cellular mRNAs are stable after YoeB induction—In spite of the abrupt inhibition of protein synthesis by YoeB induction, cellular mRNAs are much more stable after YoeB induction than after MazF induction (FIG. 9). Most importantly, full-length lpp mRNA very quickly disappears after MazF induction (within 5 min), while substantial amount of full-length lpp mRNA is present after more than 1 h of YoeB induction. Similar results were obtained using two unrelated mRNAs, ompA and rpsA mRNAs (data not shown), We have also carried out the experiment with M. tuberculosis YoeB—which is also highly toxic in E. coli—and similar to E. coli YoeB, it did not completely cleave the lpp mRNA at 90 min after induction.
4. YoeB binds to the translation initiation complex—The toeprinting experiment demonstrated that the addition of YoeB caused the toeprint band to shift by 11 bases upstream of the normal toeprinting band (13-14 bases downstream of the initiation codon). This band could is observed only in the presence of ribosomes (FIG. 10). Notably, under the same conditions, mRNA was not cleaved by YoeB in the absence of ribosomes (lane 2, FIG. 10).
5. YoeB is a 50S ribosome associating protein—Since YoeB binds to the translation initiation complex (FIG. 10), we next examined whether YoeB specifically associates with one of the ribosomal subunits. We prepared ribosome enriched extracts from cells overexpressing YoeB, purified the ribosomes over a sucrose density gradient and observed that YoeB cosediments with fractions containing the intact 70S ribosomes plus fractions containing the 50S ribosomal subunits (FIG. 11). Therefore, YoeB specifically associates with the SOS, and not the 30S subunit of ribosomes in vivo. Furthermore, the fact that YoeB binds to 70S ribosomes indicates that it does not inhibit interaction between 30S subunits and 50S subunits.
6. YoeB specifically blocks in vivo primer extension a few bases downstream of the initiation codon—Our hypothesis that YoeB inhibits the translation initiation by binding to the translation initiation complex predicts that YoeB induction causes accumulation of full length mRNAs and thus primer extension will be blocked in the vicinity of the translation initiation codon but not at any other positions in an mRNA. As seen from FIG. 12, primer extension was blocked in ompA and ompF mRNAs a few bases downstream of the initiation codon, and importantly, no other bands were detected either upstream or downstream of the initiation codon. This suggests that YoeB indeed specifically blocks translation initiation, but does not function as an endoribonuclease, which would have shown cleavage upstream and downstream of the initiation codon.

In summary—YoeB is specific protein synthesis inhibitor in prokaryotes, which binds to 50S ribosomes. We speculate that the apparent endoribonuclease activity observed in vivo (Christensen et al., 2004) and in vitro (Kamada and Hanaoka, 2005) is the intrinsic property of YoeB, which is detected only after prolonged induction of YoeB or when RNAs are incubated with a large amount of YoeB in vitro. We will continue to investigate the precise molecular mechanism of interaction of YoeB with ribosomes, which results in inhibition of translation initiation.

### Experimental Design and Methods

Our results clearly show that YoeB is a new type of toxin. We have not yet identified the exact cellular target and the molecular mechanism of inhibition of translation initiation by YoeB. We will continue to work on YoeB to achieve this goal.

### Identification of the Cellular Target of YoeB

We will use the following two different approaches: Yeast two-hybrid system which is routinely used in our laboratory to identify protein-protein interactions will be used to search for a protein or proteins interacting with YoeB in E. coli. In the second approach, we will initiate collaboration with Dr. Daniel Wilson (Max-Planck Institute for Molecular Genetics) an expert in cryo-electron microscopy, with whom we are currently collaborating to identify the location of Der (an essential GTPase in E. coli) on 50S ribosomal subunits. In addition, we will also use an E. coli cell-free system (Promega) to confirm that YoeB specifically inhibits translation initiation, but not translation elongation using well-defined synthetic homopolymers such as polyU. PolyU is used in the cell-free system to synthesize polyphenylalanine, which does not require tRNA^{fMet}, as polyU does not have the initiation codon. If our hypothesis is correct, YoeB will not inhibit the polyPhe synthesis.

### Use of the YoeB-YefM Complex for Chemical Screening

For the screening of chemicals to inhibit the YoeB-YefM complex formation, we will use two independent approaches; one approach exploits its weak intrinsic endoribonuclease activity responsible for cleavage at purine-rich sequences, (Kamada and Hanaoka, 2005) and the other uses GFP-fusion technology as described in Study 3. For the former approach, we will develop a CBS substrate containing a purine-rich YoeB cleavage sequence as shown by Kamada and Hanaoka (Kamada and Hanaoka, 2005). The CBS substrate will be synthesized as described in Study 1.

### Characterization of the Cellular Target of Doc

1. Stabilization of cellular mRNAs—The Doc-Phd TA operon has been cloned from phage P1 and expressed well using a T7 expression system (FIG. 6). Since the complex is readily prepared in a large quantity, we have initiated collaboration with Dr. John Hunt, Columbia University to determine its X-ray structure. Since its homologue exists in human pathogens such as V. cholerae (29% identity and 47% homology), screening of chemicals for this TA system has important medical relevance. In addition, our preliminary results to date reveal that this toxin is a very potent growth inhibitor by inhibiting protein synthesis at the level of translation elongation. Most significantly, as seen from FIG. 13, the cellular mRNAs are not degraded even 120 min after the induction of Doc.
2. Potent inhibitor of translation elongation—it seems that Doc functions similar to chloramphenicol or hygromycin, both of which are known to stabilize polysomes in the cells by blocking cellular mRNAs degradation. Indeed, the polysome pattern after 2 h Doc induction did not change even without the addition of hygromycin [right panel in FIG. 14; compare the upper panel (with hygromycin) with the lower panel (without hygromycin)]. On the other hand, in the absence of Doc induction, polysomes disappeared if hygromycin was not added (lower panel of the left panel in FIG. 14). This clearly indicates that Doc toxin inhibits translation elongation in a manner similar to that of chloramphenicol and hygromycin.

### Experimental Design and Methods

We will further investigate this novel protein synthesis inhibitor. We are currently preparing antiserum against Doc protein, which will be used to identify the ribosome subunit interacting with Doc. Determination of the X-ray structure of the Doc-Phd complex in collaboration with Dr. John Hunt will be highly informative regarding the interaction between Doc and Phd, and provide an insight into its cellular toxicity. We will also initiate collaboration with Dr. Daniel Wilson to determine the exact site of Doc interaction on ribosomes. As discussed for YoeB, we will also use the cell-free system to confirm that Doc is a very potent elongation inhibitor for protein synthesis irrespective of mRNAs used.

Characterization of YafQ in the YefQ Din J complex from E. coli
1. General growth inhibitor for both prokaryotes and eukaryotes—Interestingly, as shown in FIG. 7, YafQ functions as a growth inhibitor not only for E. coli but also for yeast like MazF, while YoeB or RelE are prokaryote-specific growth inhibitors. These results indicate that YafQ has a distinctly different mechanism of action from that of YoeB or RelE because its target is conserved from bacteria to eukaryotes.
2. YafQ is a sequence-specific MIase—Our preliminary data indicate that YafQ is another MIase, a sequence-specific endoribonuclease, in addition to MazF and ChpBK (FIG. 15). E. coli BW25113 cells were cotransformed with an arabinose inducible YafQ plasmid along with an IPTG inducible plasmid that expresses a nonspecific gene (in this case the era gene) to determine if YafQ induction results in enhanced cleavage of the era mRNA at specific sites relative to the control (which only expresses YafQ from the native chromosomal copy of the gene). The result shown below indicates that similar to MazF, YafQ recognizes an ACA sequence, however, it still remains to be determined if this MIase recognizes any other specific sequences.
3. DinJ and YafQ form a complex—We used affinity chromatography to demonstrate that YafQ forms a stable complex with DinJ (FIG. 16). This expression system is currently being utilized to prepare samples for X-ray crystallography by our collaborator, Dr. John Hunt (Columbia University).

### Experimental Design and Methods

We will carry out detailed experiments to determine the exact specificity of the YafQ MIase activity using various natural mRNAs and synthetic RNA as we have carried out for MazF (Zhang et al., 2005a). On the basis of the cleavage specificity thus determined, we will synthesize a CBS substrate for YafQ.

### Characterization of HipA-HipB Complex of E. Coli

HipA is a highly unusual toxin because of its high molecular weight. While all the other toxins consist of approximately 100 amino acid residues, HipA from E. coli K12 consists of 440 residues. The hipB-hipA module has been implicated to play a role in persistence leading to multi-drug resistance. It is known that a certain fraction of wild-type E. coli cell population is resistant to a number of antibiotics including penicillin even in the absence of drug-resistant genes. This phenomenon called “bacterial persistence” is considered a major medical problem while treating patients with antibiotics. Persistence is linked to preexisting heterogeneity in bacterial populations (that are genetically identical), as phenotypic switching occurs between normally growing cells and “persister” cells having reduced growth rates. Interestingly, a hipA mutant strain (hipA7, G22S and D291A) increases the “persister” cell phenotype against a number of different antibiotics (Moyed and Bertrand, 1983). Identification of the cellular target for HipA may provide important insights into the molecular mechanism of the persistence phenotype.

### Experimental Design and Methods

The HipA-HipB complex has been already well expressed in E. coli in our laboratories (FIG. 6). X-ray structural analysis of this complex has been initiated (in collaboration with Dr. John Hunt, Columbia University). In order to identify the cellular target of HipA, we will first use the yeast two-hybrid system and also attempt to isolate a cellular factor(s) that may be interacting with HipA by a pull-down experiment with use of His-tagged HipA on Ni-NTA resin. Further characterization of HipA will be dependent on the cellular target identified above. Since HipA7 mutant protein does not have lethal effect on the cells, we will express and purify this mutant protein for further biochemical characterization. We are particularly interested in the phenotype of cellular filamentation caused by HipA induction in E. coli, which suggests that HipA may be associated with cell division directly or indirectly (for example by inhibiting DNA replication). We plan to determine the cellular localization of HipA with use of antiserum against HipA, which is currently being prepared in our laboratories. Results obtained from these experiments are expected to provide important basis for further experimental approaches to solve the exact molecular mechanism by which HipA exerts its toxic effect on cell growth.

### Characterization of HigB in HigB-HigA Complex of E. Coli

The HigB-HigA complex has been already expressed (FIG. 6). We will now pursue identification of the cellular target of HigB by the methods described above for YoeB, Doc, YafQ and HipA. As this system is one of the major TA systems in the prokaryotes, the HigB-HigA complex will also be included for the screening for small molecules as described in Study 3.

Characterization of MazF Homologues from Gram Positive Bacteria

As discussed earlier, screening for small molecules for MazF homologues from B. subtilis (YdcE) and S. aureus has an important implication in developing new antibiotics for Gram-positive pathogens such as B. anthracis and S. aureus. Therefore, we will clone and express their TA complexes for Study 3. The RNA cleavage specificity for YdcE has been determined by Pellegrini et al. (Pellegrini et al., 2005). The RNA cleavage specificity of MazF homologue from S. aureus will be determined similarly as carried out for E. coli MazF (Zhang et al., 2003b).

Characterization of VapC in the VapC— Complexes in H. influenzae and M. tuberculosis

We have already cloned and expressed the VapC-VapB complex from H. influenzae (FIG. 6). We also found that the expression of H. influenzae VapC is lethal in E. coli (not shown). At present, its cellular target is not known.

### Experimental Design and Methods

Characterization of H. influenzae VapC—We have already cloned and expressed the VapC-VapB complex from H. influenzae (FIG. 6). We also found that the expression of H. influenzae VapC has lethal effect on E. coli (not shown). However, in a liquid culture, cell growth continues for a number of generations forming elongated cells (not shown). This suggest that DNA replication may be inhibited by VapC, as inhibition of DNA replication is known to block cell division resulting in the formation of filamentous cells. In this application, we will first identify the cellular target of VapC in vivo by examining the effects of VapC induction on the incorporation of uracil for RNA, thymidine for DNA and methionine for protein synthesis as described in our paper on the characterization of MazF (Zhang et al., 2003b). We will also use the yeast two hybrid system to identify the protein(s), which interacts with VapC in the cells. Since we will express the VapC-VapB complex with a His tag at the C-terminal end of VapC, we will also attempt to isolate a cellular factor(s) that may interact with VapC by a pull-down experiment with use of Ni-NTA resin. Further characterization of VapC will be dependent on the cellular target of VapC.

VapC homologues from M. tuberculosis—M. tuberculosis contains unusually a large number (23) of VapC-VapB homologues. Their phylogenetic relationships are shown in FIG. 18. Since these modules may play important roles in the dormancy of this pathogen in human tissues, it is worth targeting these complexes for screening of small molecules. This pathogen also has 9 MazF homologues, all of which have been cloned in our laboratories. Some of them were well expressed in E. coli and their MIase activities have been characterized (a manuscript is under review). Therefore, we do not anticipate any problems in cloning and expressing of these VapC-VapB modules. We will select six of them from different branches from the phylogenetic tree (mt-3, rnt-7, ret-16, mt-18 and mt-22), which wilt be cloned and expressed in E. coli. We will characterize their toxicity on the basis of the results obtained with H. influenzae VapC as described above. Their TA complexes will be expressed as GFP-fusion proteins for screening for small molecules as described in Study 3.

### Study 3: Development of a Highly Sensitive General Method to Detect Dissociation of the TA Complexes

Dissociation of the toxin-antitoxin complexes by small chemicals may be detected by measuring GFP fluorescent signals generated from GFP-tagged antitoxins in solution after removing His-tagged toxins using Ni-NTA Magnetic Agarose Beads.

GFP fusion technology has become an indispensable tool in biochemical research. However, it has been shown that a GFP-fusion protein requires a proper linker sequence between GFP and a target protein to retain the function of the target protein. Therefore, it is essential for each fusion protein to be designed to have a linker of different lengths for optimal function of the protein. For the application, GFP fusion should not inhibit the complex formation between antitoxin and toxin. For this reason, we have developed a linker library containing the linkers with a wide range of lengths. Using this library, we can identify the optimal size of a linker for each GFP-fusion TA complex.

### Rationale

Since cellular targets of most of the toxins isolated above have not yet been identified, the methods of this invention are general methods applicable to all TA systems. It is essential to establish conditions to detect dissociated antitoxins from the TA complexes in a highly sensitive manner. Accordingly, a method of this invention is a screening method with use of GFP- and His-tags.

### Experimental Approaches

Construction of an NdeI-less GFP gene—Green Fluorescent Protein (GFP) is a protein that fluoresces spontaneously. GFP can be expressed in any organism and retains its characteristic fluorescence excitation and emission properties. Since it has been shown to be extremely stable and thus readily tolerates protein fusions to either its N- or C-terminal end, it is widely used as a reporter gene to monitor expression patterns when it is fused to a protein of interest.

A mutated GFP gene in a plasmid pcDNA3-1NT-GFP-TOPO (Invitrogen) will be used, since this GFP gene has been generated by three cycles of DNA shuffling, resulting in (1) high solubility in E. coli, and (2)>40-fold increase in fluorescence over wild-type GFP. Furthermore, the codon usage of this GFP gene is optimized for expression in E. coli. This GFP protein will subsequently be referred to as Cycle-3-GFP. Cycle-3-GFP gene contains one NdeI site (at base 235 to 240; base 1 is the first base of the GFP coding sequence). We will first introduce a point mutation into the GFP gene to remove the NdeI site (CATATG→CACATG) without altering its amino acid sequence by site-directed mutagenesis using pcDNA3-1NT-GFP-TOPO plasmid as template. The resultant plasmid will be designated as pGFP(ΔNdeI). Note that the GFP gene of pcDNA3-1NT-GFP-TOPO plasmid does not contain stop codon after its coding sequence.

Construction of pET-based plasmids having His- and GFP-tags—The GFP gene will be amplified by PCR using pGFP(ΔNdeI) plasmid as template (FIG. 19). The PCR product will be introduced into pET21 plasmid (Novagen) digested with NdeI and EcoRI (FIG. 20A) and into pET28 plasmid (Novagen) digested with EcoRI and NotI (FIG. 20B). The resultant plasmids will be designated as pET21-GFP/His and pET28-His/GFP, having a His-tag sequence at downstream and upstream of the GFP sequence, respectively. Note that stop codon (TAA) will be introduced after the GFP coding sequence of pET28-His/GFP plasmid to terminate its translation.

Construction of pET-based plasmids having a His-tagged toxin gene and a GFP-tagged antitoxin gene and vice versa—We will construct pET-based expression plasmids using several TA operons derived from different organisms. In general, toxin genes are located downstream of their antitoxin genes in their operons. However, there are several exceptions with different location of toxin-antitoxin, such as the higB-higA operon in which higA (antitoxin) is located downstream of higB (toxin). Since we do not know whether His-tag or GFP-tag would be ideal to construct a fusion protein to retain its intact feature of a toxin-antitoxin complex formation, we will construct (1) His-antitoxin/toxin-GFP and GFP-antitoxin/toxin-His for the general TA operons (in the order—antitoxin-toxin; e.g. hipB-hipA, dinJ-yafQ, yefM-yoeB, relB-relE, phd-doc, vapB-vapC, ydcD-ydcE, and mazE-mazF homologue of S. aureus), and (2) His-toxin/antitoxin-GFP and GFP-toxin/antitoxin-GFP for the oppositely oriented TA operons (in the order-toxin-antitoxin; e.g. higB-higA Note that the E. coli mazE-mazF, and chpBI-chpBK genes are omitted from these constructions (see Study 1).

For each TA operon we will design two pairs of PCR primers, with EcoRI/NotI and NdeI/EcoRI sites. Using these primers, each TA operon will be amplified and cloned into both pET21-GFP/His and pET28-His/GFP plasmids digested by EcoRI/NotI and NdeI/EcoRI, respectively. The resultant plasmids will be used for purification of these TA complexes.

Purification of TA complexes—BL21(DE3) strain harboring the pET-based TA expression plasmid constructed above will be incubated at 37° C. to log phase in a synthetic medium. The TA genes will be induced for 4 h with 1 mM isopropyl-thiogalactopyranoside (IPTG). Cells will be harvested by centrifugation and suspended in buffer A [50 mM NaH₂PO₄, 300 mM NaCl, 10 mM imidazole, 1 mM β-mercaptoethanol (β-ME)]. Cells will be lysed by a French pressure cell (ThermoIEC, MA) and cell debris and unbroken cells will be removed by low speed centrifugation. The supernatant will be passed through a 0.45 μm filter (Millipore) and applied onto a Ni-NTA column (QIAGEN). The column will be washed thoroughly with buffer A and the TA complex will be eluted with 150 mM imidazole in buffer A. The samples will be pooled together and dialyzed against 50 mM Tris-HCl (pH8.0) buffer containing 50 mM NaCl and 5 mM β-ME.

Quantitation of released toxin/antitoxin proteins by measuring fluorescence signals of GFP—Before developing a high throughput screening analysis, it is important to establish conditions for detecting fluorescence signals of GFP fused to toxin/antitoxin. We will use commercially available Ni-NTA Magnetic Agarose Beads (QIAGEN) to separate dissociated GFP-tagged toxin/antitoxins from their complexes. Ni-NTA Magnetic Agarose Beads are agarose beads that contain magnetic particles and have strong metal-chelating nitrilotriacetic acid (NTA) groups covalently bound to their surfaces. These are precharged with nickel and can be used for purification in single tubes or in 96-well microplates. The magnetic beads can be used in very small volumes—as little as 10 μl can be used to purify up to 10 μg protein—thus, are convenient for high-throughput micro-scale purification in 96-well format. The fluorescent properties of the GFP protein are unaffected by prolonged treatment with 6 M guanidine-HCl, 8 M urea or 1% SDS. Prolonged (48 h) treatment with various proteases such as trypsin, chymotrypsin, papain, subtilisin, thermolysin and pancreatin at concentrations up to 1 mg/ml failed to alter the intensity of GFP (Bokman and Ward, 1981). GFP is stable in neutral buffers up to 65° C., and displays a broad range of pH stability from 5.5 to 12.

Each GFP-tagged protein forms a complex with its cognate protein in a similar manner as does its non-GFP tagged counterpart. The same amount of the TA complexes bound on Ni-NTA resin will be dissociated with 8 M urea to detect the released GFP fluorescence in solution. In eppendorf tubes, TA complex bound to Ni-NTA Magnetic Agarose in buffer A will be treated with 8 M urea at room temperature for 30 min. The tubes will be put on top of a powerful magnetic NdFeB (neodymium-iron-boron) disk to pull the released GFP-tagged proteins to the bottom of the tubes (FIG. 21). The supernatant will be transferred to empty tubes and we will measure the supernatant fluorescence using a spectrophotometer by excitation at 488 nm and detection of emission at 515 nm. The sample in buffer A without urea will be used as background controls.

### SOLUTIONS TO ANTICIPATED PROBLEMS

Some of the GFP-tagged toxins/antitoxins may not form their respective TA complexes properly due to the GFP fusion. If this is the case, we will introduce an extra linker peptide between GFP and a target protein. Another concern is that GFP fusion may inactivate toxins or antitoxins. We will test these by examining the toxicity of all the GFP-fusion toxins, which will be constructed in this application by inserting them in a pBAD vector. If cells transformed with these pBAD constructs show sensitivity to added arabinose, we will conclude that GFP-fusion does not affect the toxicity of the toxin. In a similar way, we will also insert the toxin-GFP-fused antitoxin modules into the same pBAD vector. If cells transformed with these plasmids show arabinose-sensitivity, GFP-fusion to the particular antitoxin incapacitates the antitoxin's ability to interact with its cognate toxin. In this fashion, we should be able to select toxins that can be used for high throughput screening. If neither of these constructs give a satisfactory result, we will attempt the following approaches; (1) we will extend the linker between GFP and toxin or antitoxin, which may reduce the interference of GFP with toxin or antitoxin, and (2) as a last resort, we will incorporate a cysteine residue at the C-terminal end of toxins or antitoxins, so that the TA complexes can be covalently modified with a small fluorescent molecule such as maleimide (Invitrogen).

### REFERENCES CITED

Aizenman, E., Engelberg-Kulka, H., and Glaser, G. (1996). An Escherichia coli chromosomal “addiction module” regulated by guanosine [corrected] 3′,5′-bispyrophosphate: a model for programmed bacterial cell death. Proc Natl Acad Sri USA 93, 6059-6063.
Amitai, S., Yassin, Y., and Engelberg-Kulka, H. (2004), MazF-mediated cell death in Escherichia coli: a point of no return. J Bacteriol 186, 8295-8300.
Bahassi, E. M., O'Dea, M. H., Allah, N., Messens, J., Gellert, M., and Couturier, M. (1999). Interactions of CcdB with DNA gyrase. Inactivation of Gyra, poisoning of the gyrase-DNA complex, and the antidote action of CcdA. J Biol Chem 274, 10936-10944.
Bayles, K. W. (2003). Are the molecular strategies that control apoptosis conserved in bacteria? Trends Microbiol 11, 306-311.
Bokman, S. H., and Ward, W. W. (1981). Renaturation of Aequorea green-fluorescent protein. Biochem Biophys Res Common 101, 1372-1380.
Christensen, S. K., Maenhaut-Michel, G., Mine, N., Gottesman, S., Gerdes, K., and Van Melderen, L. (2004). Overproduction of the Lon protease triggers inhibition of translation in Escherichia coli: involvement of the yefM-yoeB toxin-antitoxin system, Mol Microbiol 51, 1705-1717.
de la Cueva-Mendez, G., Mills, A. D., Clay-Farrace, L., Diaz-Orejas, R., and Laskey, R. A. (2003). Regulatable killing of eukaryotic cells by the prokaryotic proteins Kid and Kis. Embo J 22, 246-251.
Engelberg-Kulka, H., Hazan, R., and Amitai, S. (2005). mazEF: a chromosomal toxin-antitoxin module that triggers programmed cell death in bacteria. J Cell Sci 118, 4327-4332.
Engelberg-Kulka, H., Sat, B., Reches, M., Amitai, S., and Hazan, R. (2004). Bacterial programmed cell death systems as targets for antibiotics. Trends Microbiol 12, 66-71.
Gazit, E., and Sauer, R. T. (1999). The Doc toxin and Phd antidote proteins of the bacteriophage P1 plasmid addiction system form a heterotrimeric complex. J Biol Chem 274, 16813-16818.
Gerdes, K., Christensen, S. K., and Lobner-Olesen, A. (2005). Prokaryotic toxin-antitoxin stress response loci. Nat Rev Microbiol 3, 371-382.
Hargreaves, D., Santos-Sierra, S., Giraldo, R., Sabariegos-Jareno, R., de la Cueva-Mendez, G., Boelens, R., Diaz-Orejas, R., and Rafferty, J. B. (2002). Structural and functional analysis of the kid toxin protein from E. coli plasmid R1. Structure (Camb) 10, 1425-1433.
Hayes, C. S., and Sauer, R. T. (2003). Cleavage of the A site mRNA codon during ribosome pausing provides a mechanism for translational quality control. Mol Cell 12, 903-911.
Hayes, F. (2003). Toxins-antitoxins: plasmid maintenance, programmed cell death, and cell cycle arrest. Science 301, 1496-1499.
Hazan, R., Sat, B., and Engelberg-Kulka, H. (2004). Escherichia coli mazEF-mediated cell death is triggered by various stressful conditions. J Bacteriol 186, 3663-3669.
Hazan, R., Sat, B., Reches, M., and Engelberg-Kulka, H. (2001). Postsegregational killing mediated by the P1 phage “addiction module” phd-doe requires the Escherichia coli programmed cell death system mazEF. J Bacteriol 183, 2046-2050.
Kamada, K., and Hanaoka, F. (2005). Conformational change in the catalytic site of the ribonuclease YoeB toxin by YefM antitoxin. Mol Cell 19, 497-509.
Kamada, K., Hanaoka, F., and Burley, S. K. (2003). Crystal Structure of the MazE/MazF Complex. Molecular Bases of Antidote-Toxin Recognition. Mol Cell 11, 875-884.
Kampranis, S. C., Howells, A. J., and Maxwell, A. (1999). The interaction of DNA gyrase with the bacterial toxin CcdB: evidence for the existence of two gyrase-CcdB complexes. J Mol Biol 293, 733-744.
Keren, I., Shah, D., Spoering, A., Kaldalu, N., and Lewis, K. (2004). Specialized persister cells and the mechanism of multidrug tolerance in Escherichia coli. J Bacteriol 186, 8172-8180.
Korch, S. B., Henderson, T. A., and Hill, T. M. (2003). Characterization of the hipA7 allele of Escherichia coli and evidence that high persistence is governed by (p)ppGpp synthesis. Mol Microbiol 50, 1199-1213.
Li, G. Y., Zhang, Y., Chan, M. C., Mal, T. K., Hoeflich, K. P., Inouye, M., and Ikura, M. (2005). Characterization of Dual Substrate Binding Sites in the Homodimeric Structure of Escherichia coli mRNA Interferase MazF. J Mol. Biol.
Loris, R., Dao-Thi, M. H., Bahassi, E. M., Van Melderen, L., Poortmans, F., Liddington, R., Couturier, M., and Wyns, L. (1999). Crystal structure of CcdB, a topoisomerase poison from E. coli. J Mol Biol 285, 1667-1677.
Marianovsky, I., Aizenman, E., Engelberg-Kulka, H., and Glaser, G. (2001). The regulation of the Escherichia coli mazEF promoter involves an unusual alternating palindrome. J Biol Chem 276, 5975-5984.
Moyed, H. S., and Bertrand, K. P. (1983). hipA, a newly recognized gene of Escherichia coli K-12 that affects frequency of persistence after inhibition of murein synthesis. J Bacteriol 155, 768-775.
Pandey, D. P., and Gerdes, K. (2005). Toxin-antitoxin loci are highly abundant in free-living but lost from host-associated prokaryotes. Nucleic Acids Res 33, 966-976.
Pedersen, K., Zavialov, A, V., Pavlov, M. Y., Elf, J., Gerdes., K., and Ehrenberg, M. (2003). The bacterial toxin RelE displays codon-specific cleavage of mRNAs in the ribosomal A site. Cell 112, 131-140.
Pellegrini, O., Mathy, N., Gogos, A., Shapiro, L., and Condon, C. (2005). The Bacillus subtilis ydcDE operon encodes an endoribonuclease of the MazF/PemK family and its inhibitor. Mol Microbiol 56, 1139-1148.
Rice, K. C., and Bayles, K. W. (2003). Death's toolbox: examining the molecular components of bacterial programmed cell death. Mol Microbiol 50, 729-738.
Ruiz-Echevarria, M. J., Gimenez-Gallego, G., Sabariegos-Jarerto, R., and Diaz-Orejas, R. (1995). Kid, a small protein of the parD stability system of plasmid R1, is an inhibitor of DNA replication acting at the initiation of DNA synthesis. J Mol Biol 247, 568-577.
Sat, B., Hazan, R., Fisher, T., Khaner, H., Glaser, G., and Engelberg-Kulka, H. (2001). Programmed cell death in Escherichia coli: some antibiotics can trigger mazEF lethality. J Bacteriol 183, 2041-2045.
Sat, B., Recites, M., and Engelberg-Kulka, H. (2003). The Escherichia coli mazEF suicide module mediates thymineless death. J Bacterial 185, 1803-1807.
Suzuki, M., Zhang, J., Liu, M., Woychik, N. A., and Inouye, M. (2005). Single protein production in living cells facilitated by an mRNA interferase. Mol Cell 18, 253-261.
Takagi, H., Kakuta, Y., Okada, T., Yao, M., Tanaka, I., and Kimura, M. (2005). Crystal structure of archaeal toxin-antitoxin RelE-RelB complex with implications for toxin activity and antitoxin effects. Nat Struct Mol Biol 12, 327-331,
Zhang, J., Zhang, Y., and Inouye, M. (2003a). Characterization of the interactions within the mazEF addiction module of Escherichia coli. J Biol Chem 278, 32300-32306.
Zhang, 3., Zhang, Y., Zhu, L., Suzuki, M., and Inouye, M. (2004). Interference of mRNA function by sequence-specific endoribonuclease PemK. J Biol Chem 279, 20678-20684.
Zhang, Y., Zhang, 3., Hara, H., Kato, I., and Inouye, M. (2005a). Insights into the mRNA Cleavage Mechanism by MazF, an mRNA Interferase. J Biol Chem 280, 3143-3150.
Zhang, Y., Zhang, 3., Hoeflich, K. P., Ikura, M., Qing, G., and Inouye, M. (2003b). MazF cleaves cellular mRNAs specifically at ACA to block protein synthesis in Escherichia coli. Mot Cell 12, 913-923.
Zhang, Y., Zhu, L., Zhang, 3., and Inouye, M. (2005b). Characterization of ChpBK, an mRNA interferase from Escherichia coli. J Biol Chem 280, 26080-26088.

1. A method for identifying an agent which prevents or partially prevents an antitoxin from forming a complex with its cognate toxin, comprising contacting a potential agent with a labeled substrate in solution, whereby detection of the label indicates presence of an agent that prevents an antitoxin from forming a complex with a toxin.

2. The method of claim 1 used to identify agents functioning as mRNA interferases.

3. The method of claim 1, wherein the substrate comprises a short DNA-RNA chimeric substrate.

4. The method of claim 3, wherein the chimeric substrate comprises approximately 12 bases.

5. The method of claim 4, wherein the substrate is dGdAdTdArUdAdCdAdTdAdTdG (SEQ ID NO: 9) labeled by attaching a fluorescent probe at the 5′ end and a quencher at the 3′ end.

6. The method of claim 5, wherein the fluorescent probe is ROX, and the quencher is Eclipse.

7. The method of claim 5, wherein the substrate is a cleavable beacon substrate (CBS-I).

8. The method of claim 5, whereby the method is used to identify agents which prevent MazE/MazF complex formation.

9. The method of claim 4, wherein the substrate is dGdAdTdArUrArCdGdTdAdTdG (SEQ ID NO: 10) labeled by attaching a fluorescent probe at the 5′ end and a quencher at the 3′ end.

10. The method of claim 9, wherein the fluorescent probe is ROX, and the quencher is Eclipse.

11. The method of claim 9, wherein the substrate is a cleavable beacon substrate (CBS-2).

12. The method of claim 9, whereby the method is used to identify agents which prevent ChpBI/ChpBK complex formation or YdcD/YdcE complex formation.

13. The method of claim 4, wherein the substrate is dGdAdTdArUrArCdCdTdAdTdG (SEQ ID NO: 11) labeled by attaching a fluorescent probe at the 5′ end and a quencher at the 3′ end.

14. The method of claim 13, wherein the fluorescent probe is ROX, and the quencher is Eclipse.

15. The method of claim 13, wherein the substrate is a cleavable beacon substrate (CBS-3).

16. The method of claim 13, whereby the method is used to identify agents which prevent YdcD/YdcE complex formation.

17. The method of claim 1, wherein the substrate comprises a GFP-tagged antitoxin and His-tagged toxin or a His-tagged antitoxin and GFP-tagged toxin.

18. The method of claim 17, wherein the GFP-tagged toxin or GFP-tagged antitoxin contain a linker between the GFP and the toxin or between the GFP and the antitoxin.

19. The method of claim 17 used for detecting agents not functioning as mRNA interferases.

20. The method of claim 17, wherein the labeled AT complex substrate, if dissociated, is detected by measuring GFP fluorescent signals generated from GFP-tagged antitoxins in solution after removing His-tagged toxins using Ni-NTA Magnetic Agarose Beads.

21. The method of claim 17, wherein the labeled AT complex substrate, if dissociated, is detected by measuring GFP fluorescent signals generated from GFP-tagged toxins in solution after removing His-tagged antitoxins using Ni-NTA Magnetic Agarose Beads.

22. An agent identified by the method of claim 1.

23. An agent capable of interfering with formation of a toxin-antitoxin complex.

24. The agent of claim 22, wherein the toxin-antitoxin complex is in a bacterial cell.

25. A composition comprising one or more different agents of claim 22 in combination with one or more different conventional antibiotics.

26. A pharmaceutical composition comprising the composition of claim 25 additionally comprising pharmaceutical excipients.

27. A method for killing or inhibiting growth of microbial cells comprising contacting the microbial cells with an agent of claim 22.

28. A method of treating an infection comprising administering the pharmaceutical composition of claim 26.

29. The method of claim 28, wherein the infection is tuberculosis.

30. The method of claim 28, wherein the infection is caused by antibiotic-resistant bacteria.

31. The method of claim 30, wherein the antibiotic-resistant bacteria are resistant to vancomycin.

32. The method of claim 27, wherein the microbial cells are pathogens used for bioterrorism.

33. A method of regulating bacterial cell dormancy comprising contacting the cell with an agent of claim 22 to cause the cell to become dormant instead of causing the cell to die.
